(19) United States
(12) Patent Application Publication
(10) Pub. No.: US 2025/0037884 A1
(43) Pub. Date: Jan. 30, 2025

(54) CYBER-PHYSICAL SYSTEM TO ENHANCE USABILITY AND QUALITY OF TELEHEALTH CONSULTATION

(71) Applicants: THE GEORGE WASHINGTON UNIVERSITY, Washington, DC (US); Care Constitution Corp., Newark, DE (US)

(72) Inventors: Marc P. GARBEY, Houston, TX (US); Guillaume JOERGER, Strasbourg (FR)

(73) Assignees: THE GEORGE WASHINGTON UNIVERSITY, Washington, DC (US); Care Constitution Corp., Newark, DE (US)

(21) Appl. No.: 18/791,292

(22) Filed: Jul. 31, 2024

Related U.S. Application Data
(63) Continuation of application No. PCT/US2023/061783, filed on Feb. 1, 2023.
(60) Provisional application No. 63/305,420, filed on Feb. 1, 2022.

(51) Int. Cl. G16H 80/00; A61B 5/00; G16H 10/20; G16H 10/60; G16H 50/20

(52) U.S. Cl. CPC G16H 80/00; A61B 5/0002; A61B 5/4088; A61B 5/7264; A61B 5/742; G16H 10/20; G16H 10/60; G16H 50/20

(57) ABSTRACT

A cyber-physical system for conducting a telehealth session. In embodiments, the system includes a hardware control box that enables patients (e.g., elderly or cognitively-impaired patients) to easily participate in the telehealth session. In some embodiments, the system analyzes sensor data (e.g., thermal images, eye tracking data, etc.) and calculates state variables that form a mathematical representation (a “digital twin”) indicative of the physical, emotive, cognitive, or social state of the patient. In some of those embodiments, the system calculates Myasthenia Gravis core examination metrics by performing computer vision analysis on patient video data and/or audio analysis on patient audio data. In some embodiments, the system controls a pan-tile-zoom camera to zoom in on each region of interest that is relevant to the examination. In some embodiments, the digital twin is used as an input of a heuristic computer reasoning system, which uses artificial intelligence to support clinical diagnosis and decision-making.

### CROSS-REFERENCE TO RELATED APPLICATIONS

This application is a continuation application of International Application No. PCT/US2023/061783, filed Feb. 1, 2022, which claims priority to U.S. Prov. Pat. Appl. No. 63/305,420, filed Feb. 1, 2022, which is hereby incorporated by reference.

### FEDERAL FUNDING

This invention was made with government support under Grant No. U54 NS115054 awarded by NIH. The U.S. government has certain rights in the invention.

### BACKGROUND

Telemedicine enables practitioners and patients (including disabled patients who have difficulty traveling to in-person consultations) to interact at anytime from anywhere in the world, reducing the time and cost of transportation, reducing the risk of infection by allowing patients to receive care remotely, reducing patient wait times, and enabling practitioners to spend more of their time providing care patients. Accordingly, telemedicine has the potential to improve the efficiency of the medical consultations for patients seeking medical care, practitioners evaluating the effectiveness of a specific treatment (e.g., as part of a clinical trial), etc.

Telemedicine also provides a platform for capturing and digitizing relevant information and adding that data to the electronic health records of the patient, enabling the practitioner to for example, using voice recognition and natural language processing to assist the provider in documenting the consultation and even recognizing the patient pointing to a region of interest and selecting a keyword identifying that region of interest¹).

However, telemedicine has a number of drawbacks. Practitioners using existing telemedicine systems must rely on two-dimensional images and audio that are often low resolution, filtered, and compressed. With traditional telemedicine systems, the practitioner's view of the patient is limited and outside of the practitioner's control. Meanwhile, practitioners also cannot control other aspects of the patient environment, such as lightening, equipment, environmental distractions, etc. Finally, practitioners often find it difficult to simultaneously conduct the telemedicine consultation and document the exam without diminishing the quality of communication. In particular, keeping eye contact, mental focus, and maintaining effective listening require a large amount of the doctor's attention.

From the patient standpoint, many patients report that telehealth consultations yield lower quality of care compared to in-person visits, that health care providers are not able to conduct a physical exam at all, that they have difficulty seeing or hearing health care providers, that they feel less personally connected to the health care providers during telehealth visits, and/or that they have privacy concerns about telemedicine. The draw backs of telemedicine are particularly acute for elderly or cognitively impaired patients, who may have difficulty using—or may not have access to—the computing devices required to connect to existing telemedicine systems.

Accordingly, there is a need for an improved system to enhance the usability and quality of telehealth communication. In particular, there is a need for a system that enables patients-particularly elderly and cognitively-impaired patients-to easily participate in telehealth sessions. Additionally, there is a need for a telehealth platform that noninvasively captures (and digitizes) information indicative of the physical, emotive, cognitive, and/or social state of the patient.

### SUMMARY

Disclosed is a cyber-physical system (e.g., a practitioner system and a patient system) for conducting a telehealth session between a practitioner and a patient. In some embodiments, the patient system includes a hardware control box that enables patients-including elderly or cognitively-impaired patients-to easily initiate the telehealth session (e.g., with a single click of a hardware button or by saying a simple voice command) without using any software application, touch display, keyboard, or mouse. In those embodiments, the system is particularly well suited for conducting a computer-assisted cognitive impairment assessment, for example by outputting questions for the patient, providing functionality for the patient to easily answer those questions using the hardware buttons on the control box, time stamping the questions and patient responses, and calculating variables indicative of the cognitive state of the patient based on time-stamped questions and the time-stamped responses.

In some embodiments, the patient system includes environmental sensors, enabling the practitioner to view and assess environmental conditions (e.g., temperature, humidity, airborne particles, etc.) that may be affecting the health conditions of the patient.

In some embodiments, the system analyzes sensor data captured by the patient system (e.g., thermal images captured by a thermal imaging camera, eye tracking data captured by an eye tracker, three-dimensional images captured by a depth camera, etc.) and calculates state variables indicative of the physical, emotive, cognitive, or social state of the patient. For example, to calculate state variables (e.g., the Myasthenia Gravis core examination metrics), a computer vision module may perform computer vision analysis on patient video data and/or an audio analysis module may perform audio analysis on patient audio data.

In some embodiments, the state variables calculated by the system, together with the electronic health records of the patient and subjective assessments of the practitioner, form a “digital twin”—a mathematical representation of the physical, emotive, cognitive, and/or social state of the patient. In some of those embodiments, the digital twin may be used as an input of a heuristic computer reasoning system, which uses artificial intelligence to support clinical diagnosis and decision-making. For example, the heuristic computer reasoning engine may detect deviations from previously-determined state variables or identify potentially relevant diagnostic explorations.

The patient system includes a patient camera for capturing images of the patient. In some embodiments, the patient camera may be inside a camera enclosure that prevents the patient from seeing the patient camera (while still allowing the patient camera to capture images of the patient), to prevent the patient camera from distracting the patient and allow the patient to focus on the dialog with the practitioner. In some embodiments, the patient camera is a remotely-controllable pan-tile-zoom (TPZ) camera that can be controlled remotely (e.g., by the practitioner or automatically by the system) to capture images of a region of interest that is relevant to the examination being performed. In some of those embodiments, the computer vision module may use the digital twin of the patient to recognize a region of interest in the patient video data captured by the pan-tile-zoom camera and output control signals to the pan-tile-zoom camera to zoom in on the region of interest.

### BRIEF DESCRIPTION OF THE DRAWINGS

Aspects of exemplary embodiments may be better understood with reference to the accompanying drawings. The components in the drawings are not necessarily to scale, emphasis instead being placed upon illustrating the principles of exemplary embodiments.

FIG. 1 is a diagram of a cyber-physical telehealth system, which includes a practitioner system and a patient system, according to exemplary embodiments.

FIG. 2A is a diagram of the patient system, which includes a patient computing system, a camera enclosure, and a hardware control box, according to exemplary embodiments.

FIG. 2B is a diagram of the patient system according to another exemplary embodiment.

FIG. 2C is a diagram of the patient system according to another exemplary embodiment.

FIG. 3 is a diagram of the interior of the hardware control box of FIG. 2A according to exemplary embodiments.

FIG. 4 is a diagram of the exterior of the hardware control box of FIG. 3 according to exemplary embodiments.

FIG. 5 is a diagram of the patient computing system of FIG. 2A according to exemplary embodiments.

FIG. 6 is a diagram of the camera enclosure of FIG. 2A according to exemplary embodiments.

FIG. 7A is a block diagram of a videoconferencing module according to exemplary embodiments.

FIG. 7B is a block diagram of a sensor data classification module according to exemplary embodiments.

FIG. 7C is a diagram of example body landmarks.

FIG. 7D is a diagram of example face landmarks.

FIG. 7E is images of example regions of interest in patient video data according to exemplary embodiments.

FIG. 7F is a graph illustrating a stochastic run of a phenomenological model according to exemplary embodiments.

FIG. 7G is a graph illustrating the fitting the phenomenological model of FIG. 7F to the response curve for an example muscle group according to an exemplary embodiment.

FIG. 7H is a graph illustrating the fitting the phenomenological model of FIG. 7F to the response curve for another example muscle group according to an exemplary embodiment.

FIG. 7I is a block diagram of patient system controls according to exemplary embodiments.

FIG. 7J is a block diagram illustrating an audio calibration module, a patient tracking module, and a lighting calibration module according to exemplary embodiments.

FIG. 7K is a block diagram illustrating the output of visual aids to assist the patient and/or the practitioner according to exemplary embodiments.

FIG. 8A is a flowchart illustrating the construction of a digital twin according to exemplary embodiments.

FIG. 8B is a graph illustrating a stochastic process used to retrofit the digital twin according to exemplary embodiments.

FIG. 8C is a flowchart illustrating a continuous, stochastic process that for predicting patient trajectory with respect to his/her state variable with respect to time according to exemplary embodiments.

FIG. 8D is a graph illustrating an exploration forward in time, by a heuristic computer reasoning engine, simulating a change in a state variable according to exemplary embodiments.

FIG. 8E is a graph illustrating a simulation, by the heuristic computer reasoning engine, of a change in a control variable according to exemplary embodiments.

FIG. 8F is a graph illustrating a backward search, by the heuristic computer reasoning engine, to analyze the potential of causality in patient conditions according to exemplary embodiments.

FIG. 9 is a view of a practitioner user interface according to exemplary embodiments.

### DETAILED DESCRIPTION

Reference to the drawings illustrating various views of exemplary embodiments is now made. In the drawings and the description of the drawings herein, certain terminology is used for convenience only and is not to be taken as limiting the embodiments of the present invention. Furthermore, in the drawings and the description below, like numerals indicate like elements throughout.

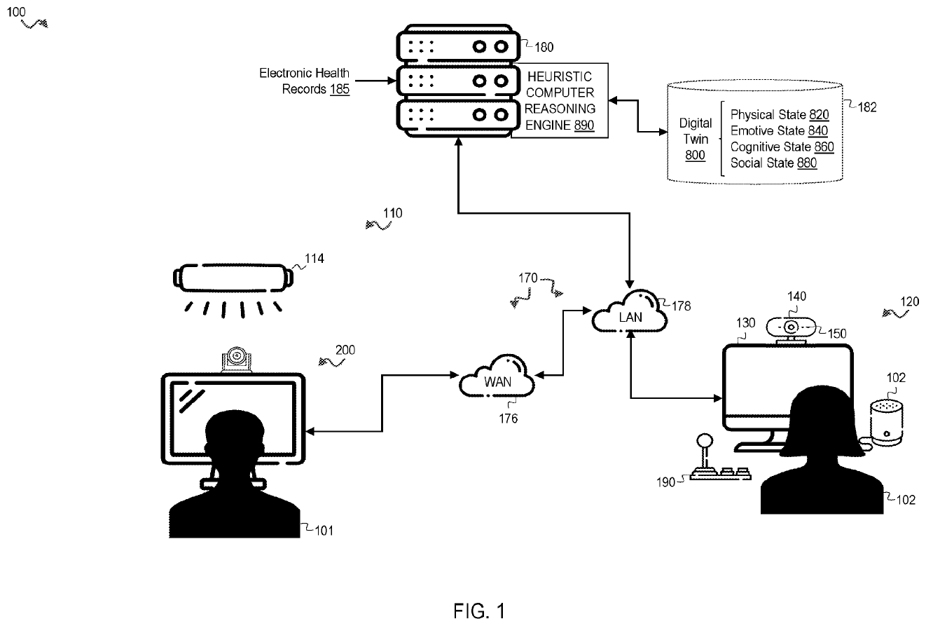

FIG. 1 is a diagram of a remotely-controllable cyber-physical telehealth system 100 according to exemplary embodiments.

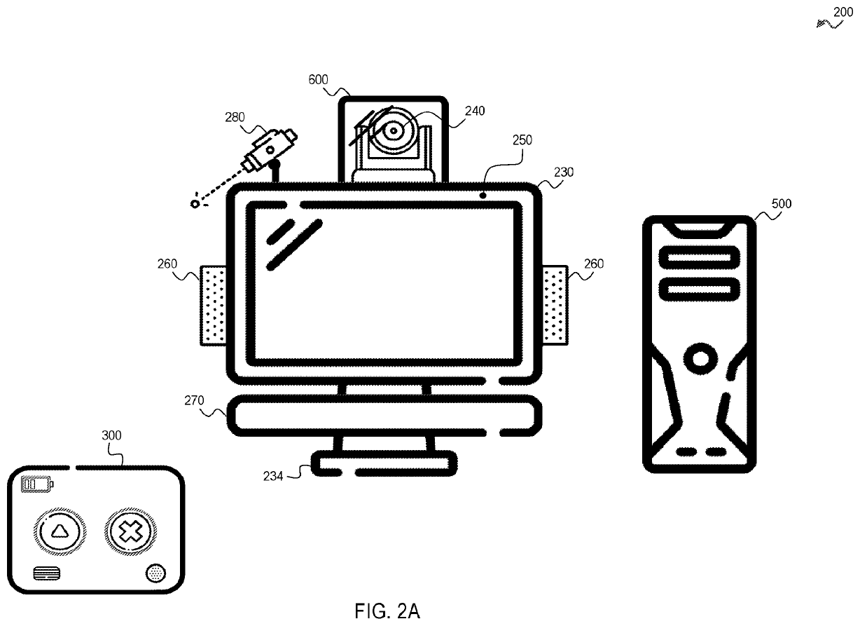

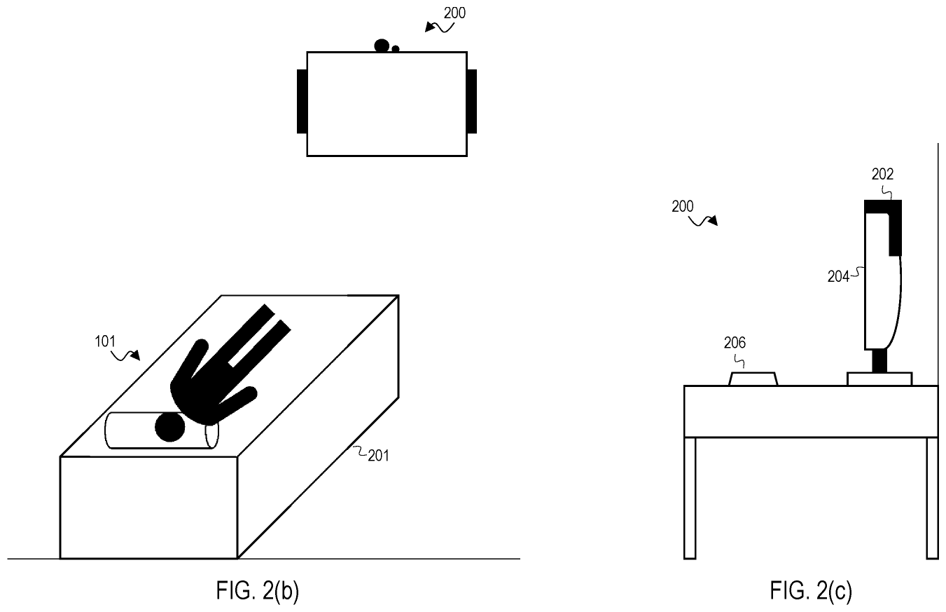

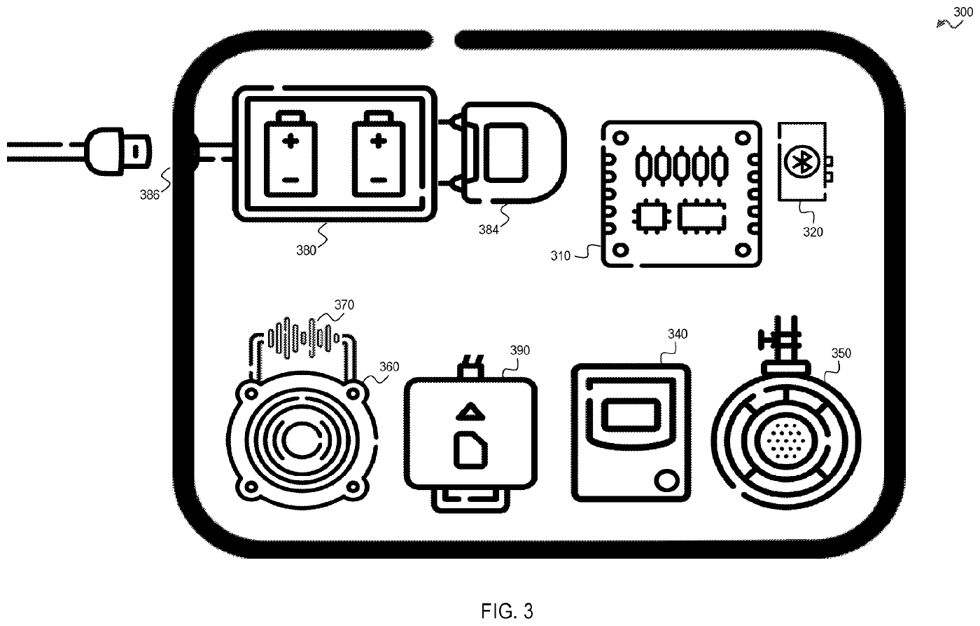

In the embodiment of FIG. 1, the cyber-physical system 100 includes a practitioner system 120 (for use by a physician or other health practitioner 102) in communication, via one or more communications networks 170, with a patient system 200 (FIG. 2(a)) and a patient computing system 500 (FIG. 2(a)) located in a patient environment 110 of a patient 101. The practitioner system 120 includes a practitioner display 130, a practitioner camera 140, a practitioner microphone 150, a practitioner speaker 160, and a patient system controller 190. In some embodiments, the patient environment 120 includes a remotely-controllable lighting system 114, which enables the brightness of the patient environment 110 to be remotely adjusted. The communications network(s) 170 may include wide area networks 176 (e.g., the Internet), local area networks 178, etc. In some embodiments, the patient computing system 500 and the practitioner system 120 are in communication with a server 180 having a database 182 to store the data from the analysis via the communications network(s) 170.

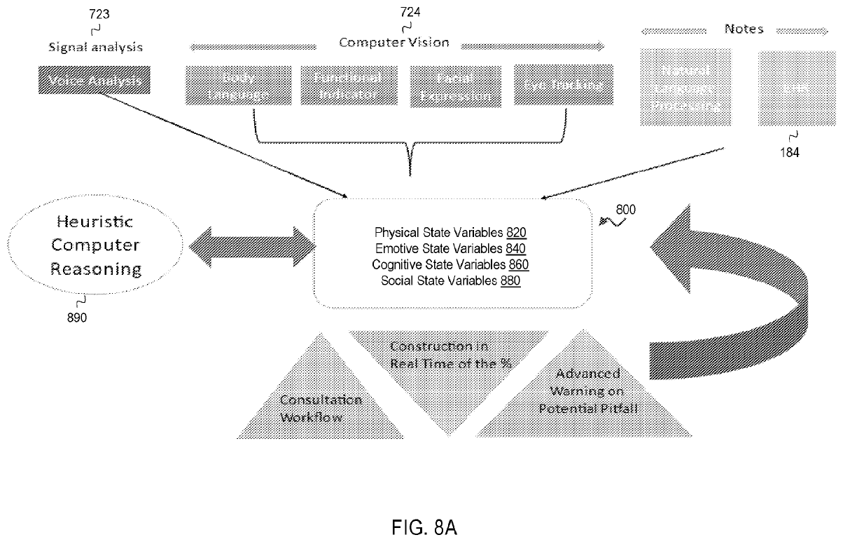

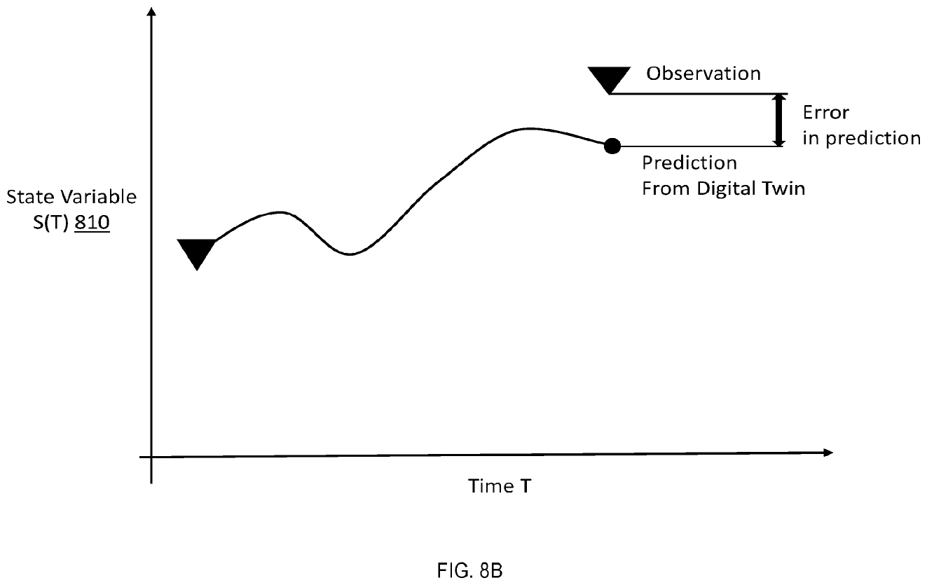

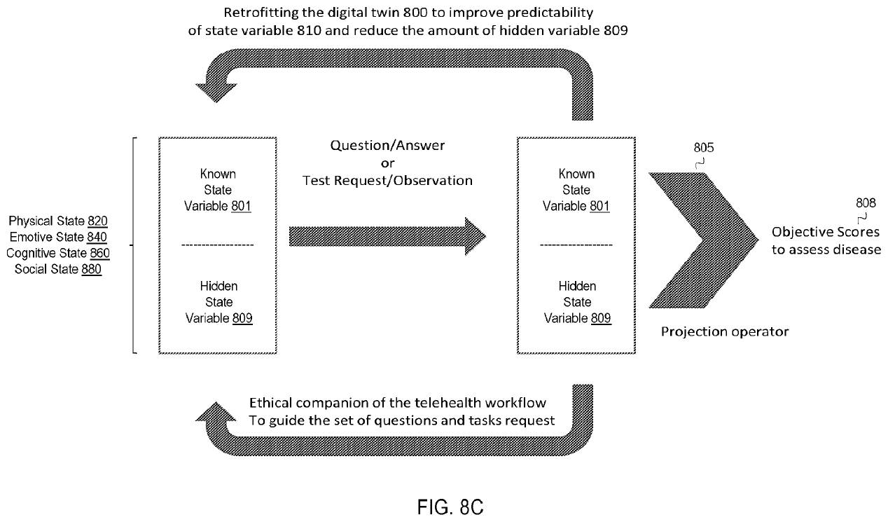

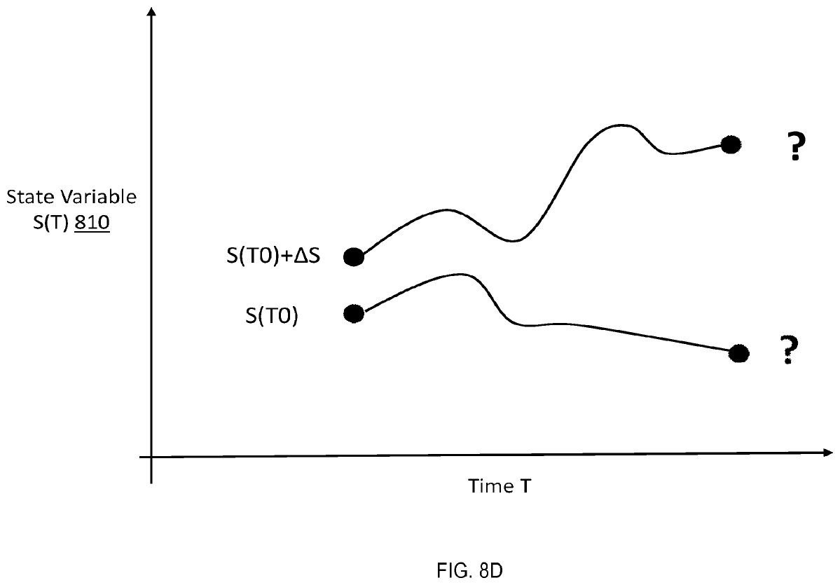

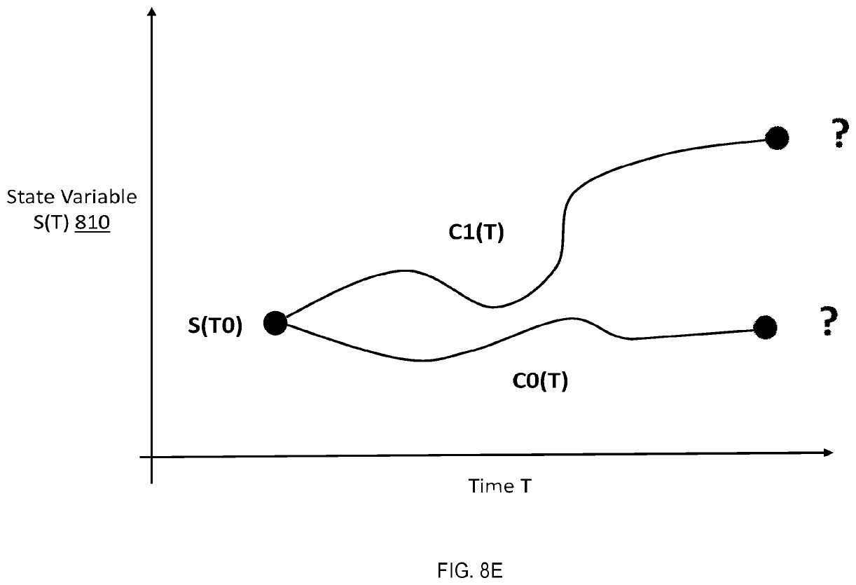

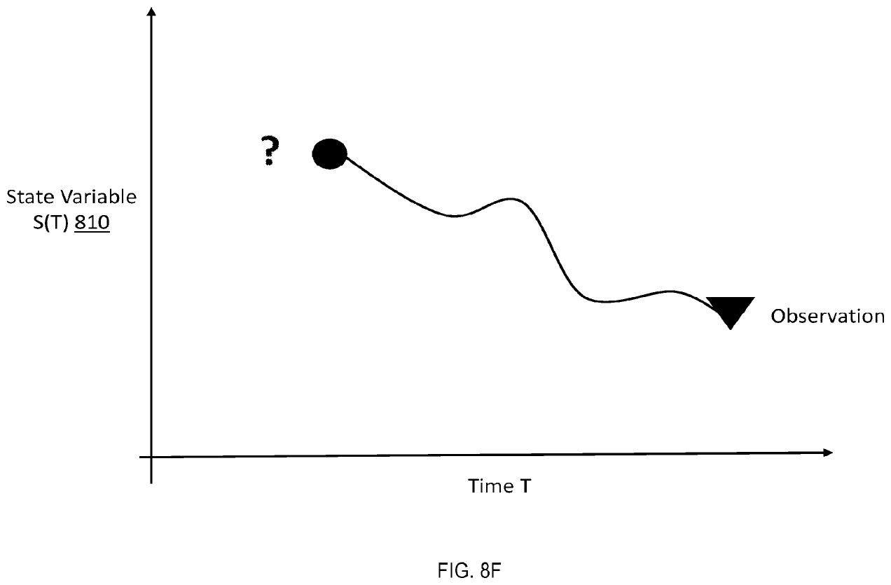

As described in detail below; the cyber-physical system 100 generates objective metrics indicative of the physical, emotive, cognitive, and/or social state of the patient 101. (Additionally, the cyber-physical system 100 may also provide functionality for the practitioner 102 to provide subjective assessments of the physical, emotive, cognitive, and/or social state of the patient 101.) Together with the electronic health records 184 of the patient 101, those objective metrics and/or subjective assessments are used to form a digital representation of the patient 101 (referred to as a digital twin 800, which is described in detail below with reference to FIG. 8A-8F) that includes physical state variables 820 indicative of the physical state of the patient 101, emotive state variables 840 indicative of the emotive state of the patient 101, cognitive state variables 860 indicative of the cognitive state of the patient 101, and/or social state variables 880 indicative of the social state of the patient 101. The digital twin 800, which is stored in the database 182, provides a mathematical representation of the state of the patient 101 (e.g., at each of a number of discrete points in time), which may be used by a heuristic computer reasoning engine 890 (described in detail below with reference to FIGS. 8B-8F) that uses artificial intelligence to support clinical diagnosis and decision-making.

FIGS. 2A through 2C are diagrams of the patient system 200 according to exemplary embodiments.

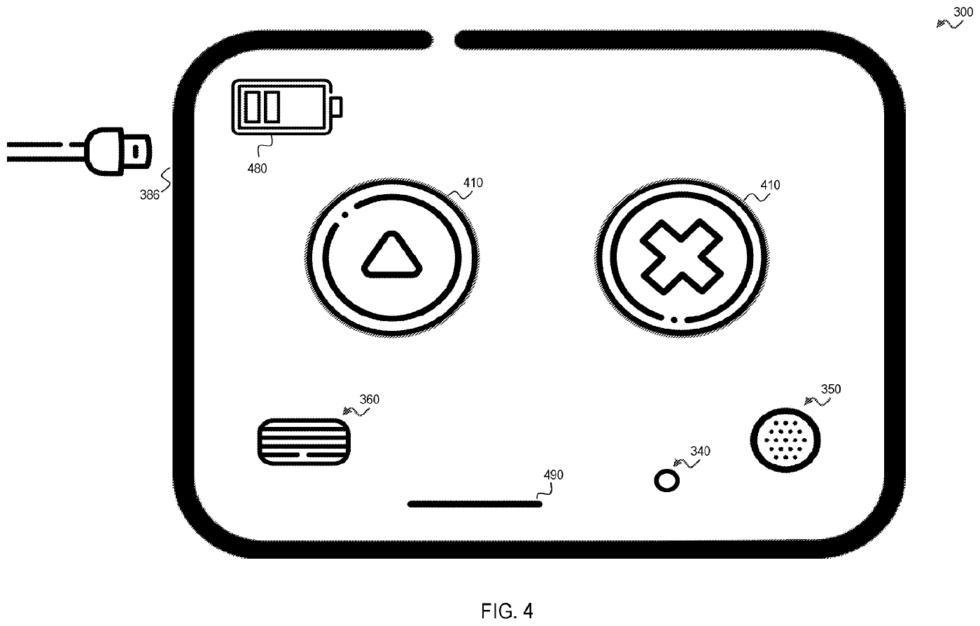

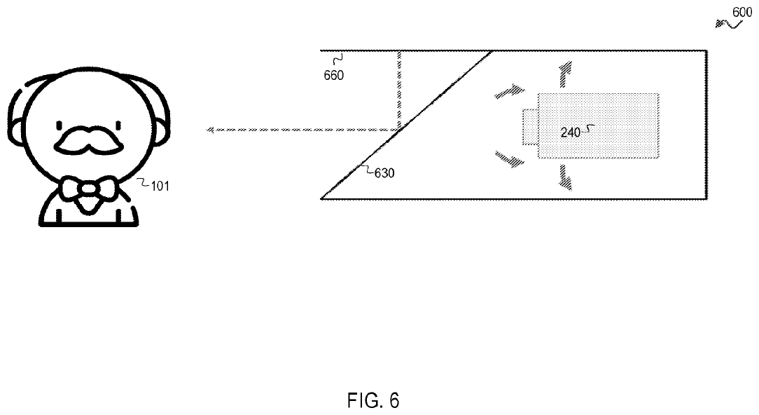

In the embodiment of FIG. 2A, the patient system 200 includes a patient display 230, a patient camera 240 (inside a camera enclosure 600, which is described below with reference to FIG. 6), a thermal imaging camera 250, speakers 260, an eye tracker 270, a laser pointer 280, and a control box 300 (described in detail below with reference to FIGS. 3 and 4). The patient camera 240 is a high definition, remotely-controllable pan-tilt-zoom (PTZ) camera with adjustable horizontal position (pan), vertical position (tilt), and focal length of the lens (zoom). In some embodiments, the patient display 230 may be mounted on a remotely-controllable rotating base 234, enabling the horizontal orientation of the patient display 230 to be remotely adjusted. Additionally, in some embodiments, the patient display 230 may also be mounted on a remotely-controllable vertically-adjustable mount (not shown), enabling the vertical orientation of the patient display 230 to be remotely adjusted.

As shown in FIG. 2B, the patient system 200 may be used in clinical settings, for example by a patient 101 in a hospital bed 201. As shown in FIG. 2C, the patient system 200 may be used in conjunction with a patient computing system 500 that includes a processing device such as, for example, a traditional desktop computer, for example having a display 204 and a keyboard 206. In those embodiments, for example, the patient system 200 may be realized as a compact system package that can be mounted on the display 204.

FIG. 3 is a block diagram of the internal components of the control box 300 according to exemplary embodiments.

In the embodiment of FIG. 3, the control box 300 includes a microcomputer 310 (e.g., a Raspberry Pi), a communications module 320, a patient microphone 350, a speaker 360, a beeper 370, a battery 380, a battery gauge 384, and a power source connection 386 (e.g., a USB port). In some embodiments, the control box 300 may also include an identification reader 390 (e.g., an electronic card reader). In some embodiments, the control box 300 may also include a physiological sensor, such as a breath sensor 340. The breath sensor 340 may include, for example, a device for estimating blood alcohol content from a breath sample, for detecting viruses or diseases from a breath sample, and/or for measuring hydrogen and/or methane content in a breath sample (e.g., a FoodMarble AIRE), etc.

FIG. 4 is an exterior view of the control box 300 according to an exemplary embodiment.

In the embodiment of FIG. 4, the control box 300 includes a first button 410 (e.g., a left button), a second button 420 (e.g., a right button), access to the patient speaker 360 (e.g., slots) and the patient microphone 350 (e.g., holes), a battery display 480 indicating the charge level of the battery 380 (as determined by the battery gauge 384), and the power source connection 386. In some embodiments, both buttons 410 and 420 have light capabilities and can be turned on patient (e.g., via the patient computer 500) or remotely by the practitioner 102 (e.g., via the patient system control 190). In some embodiments, the control box 300 may also include access to the breath sensor 340 (e.g., a tube) and/or an identification reader inlet 490 (e.g., a slot) that provides physical access to the identification reader 390.

Unlike keyboards and other generic user input devices, which enable users to provide input data for processing by any software application running on a computing system, the control box 300 may be a dedicated hardware device for users to provide input data (e.g., via the microcomputer 310) that is processed solely by the telehealth software described herein. While the control box 300 may be configured to perform multiple telehealth functions as described below (e.g., providing functionality for the patient 101 to initiate a telehealth session, capturing patient audio data via the patient microphone 350, outputting practitioner audio data via the speaker 360, providing functionality for the patient 101 to provide responses using the buttons 410 and 420, etc.), in some embodiments the control box 300 can be described as a single purpose hardware device, meaning the control box 300 is solely for use by the telehealth software described herein.

In some embodiments, the patient system 200 does not include any user input device (e.g., a keyboard, a mouse, etc.) other than the control box 300, enabling patients 101 (including elderly and/or cognitively-impaired patients 101) to easily initiate and participate in telehealth sessions as described below. In other embodiments, the patient system 200 includes the control box 300 in addition to one or more generic, multi-purpose user input devices (e.g., a keyboard, a mouse, etc.).

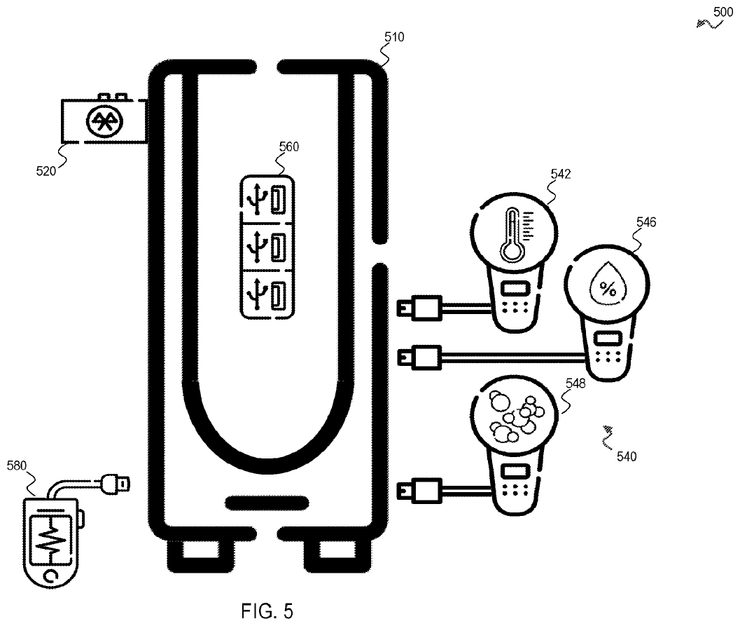

FIG. 5 is a block diagram of the patient computing system 500 according to exemplary embodiments.

In the embodiment of FIG. 5, the patient computing system 500 includes a processing device such as a compact computer 510, a communications module 520, environmental sensors 540, and one or more universal serial bus (USB) ports 560. The environmental sensors 540 may include any sensor that measures information indicative of an environmental condition of the patient environment 110, such as a temperature sensor 542, a humidity sensor 546, an airborne particle senor 548, etc. In some embodiments, the patient computing system 500 may include one or more physiological sensors 580. The physiological sensors 580 may include any sensor that measures a physiological condition of the patient 101, such as a pulse oximeter, a blood pressure monitor, an electrocardiogram, etc. The physiological sensors 580 may interface with the patient computing system 500 via the USB port(s) 560, which may also provide functionality to upload physiological data from an external health monitoring device (e.g., data indicative of the sleep and/or physical activity of the patient captured by a smartwatch or other wearable activity tracking device). Additionally or alternatively, one or more environmental sensors 540 and/or physiological sensors 580 (such as the breath sensor 340 described above) may be located in or on the control box 300.

The communications modules 320 and 520 of the control box 300 and the patient computing system 500 may be any device suitably configured to send data from the control box 300 to the patient computing system 500 via a wired connection, a wireless connection (e.g., Bluetooth), a local area network 178, etc.

FIG. 6 is a diagram illustrating the camera enclosure 600 according to exemplary embodiments.

The presence of the patient camera 240 may distract the patient 101 and prevent the patient 101 from focusing on the interaction with the practitioner 102. In particular, in embodiments where the patient camera 240 is a remotely-controllable pan-tilt-zoom (PTZ) camera, any movement of the patient camera 240 may be particularly distracting. Therefore, in the embodiment of FIG. 6, the patient camera 240 is enclosed in the camera enclosure 600 that includes a one-way mirror 630. The mirror 630 allows light to pass from the camera 240 to the patient 101, enabling the camera 240 to capture images of the patient 101. However, the mirror 630 reflects light from the patient 101 (for example, toward an interior wall 660), preventing the patient 101 from seeing the patient camera 240 and any movement of the patient camera 240.

By hiding the patient camera 240 in a “black box,” the camera enclosure 600 ensures that patient camera 240 is as minimally invasive as possible, enabling the patient 101 to focus on the dialog with the practitioner 102 dialogue between the patient 101 and the practitioner 102.

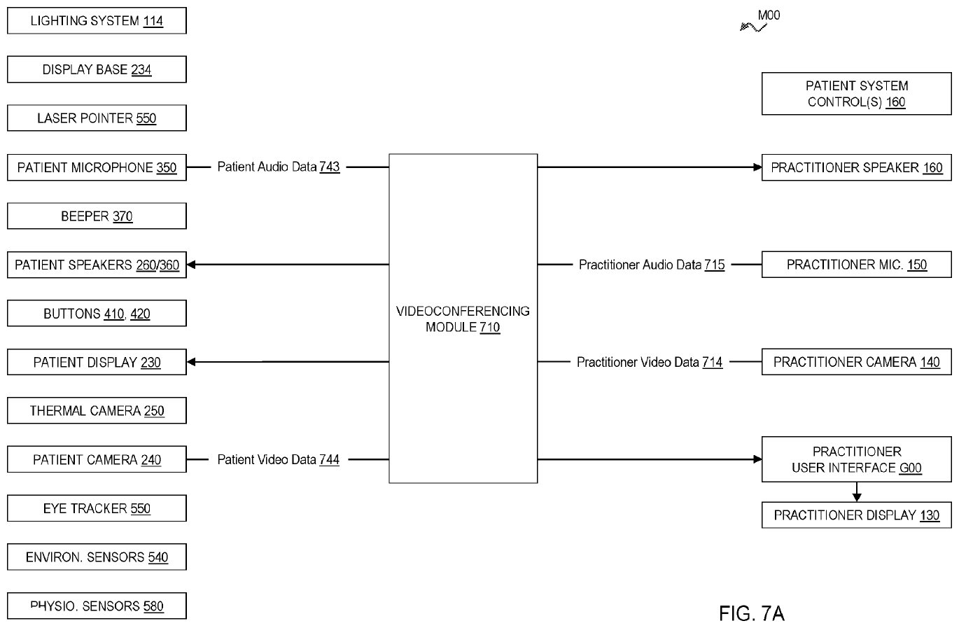

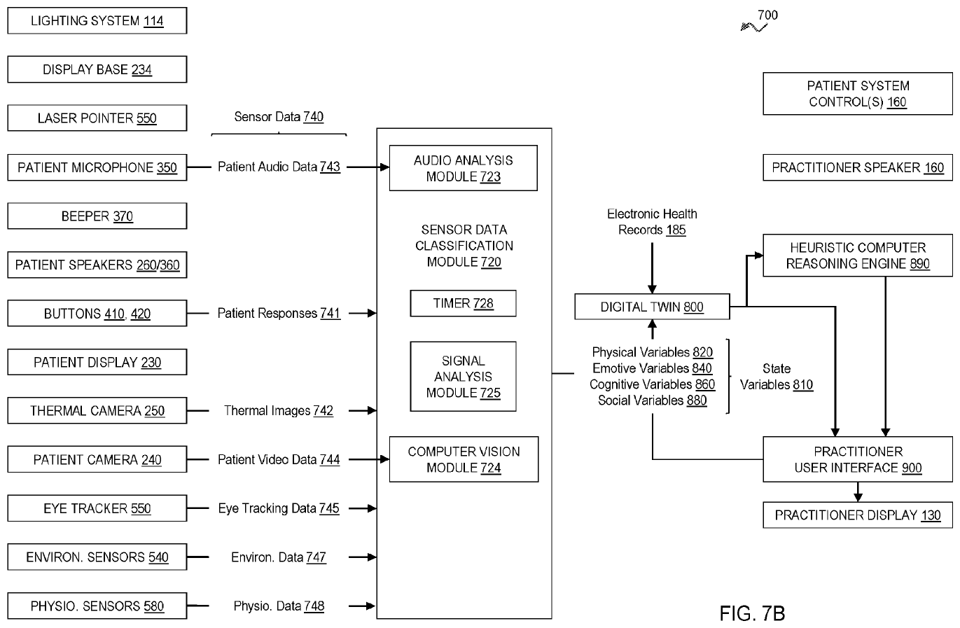

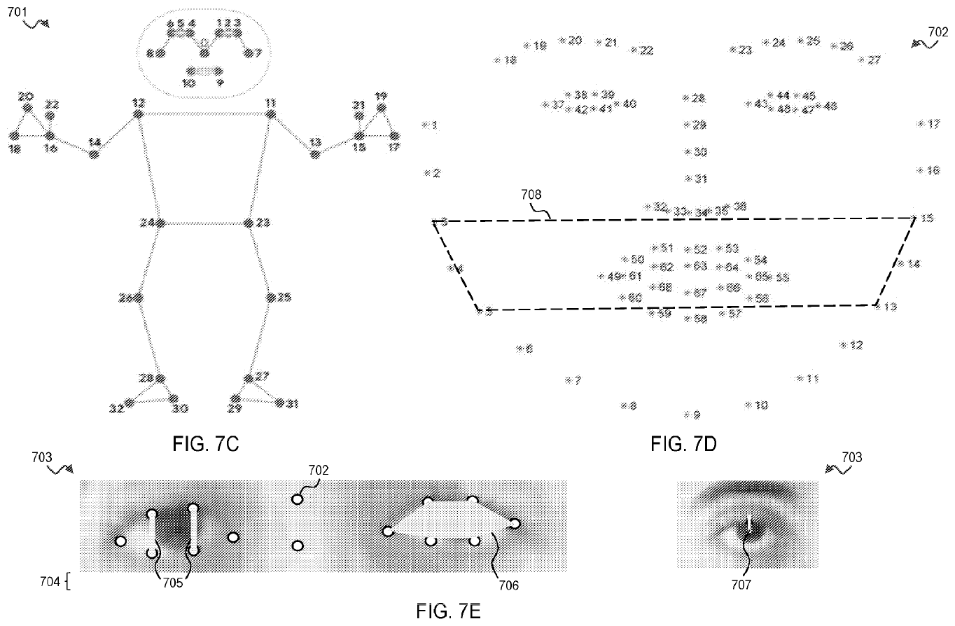

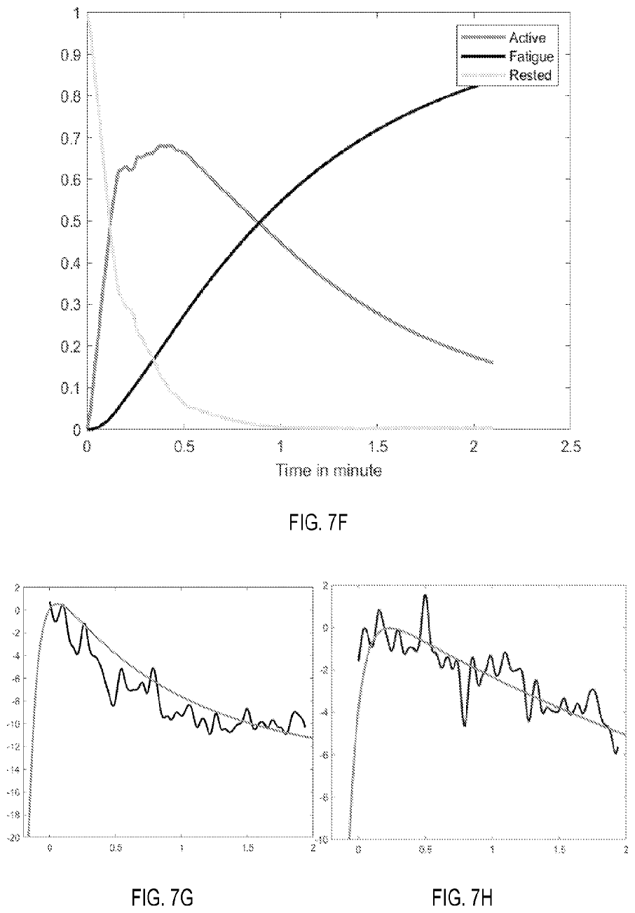

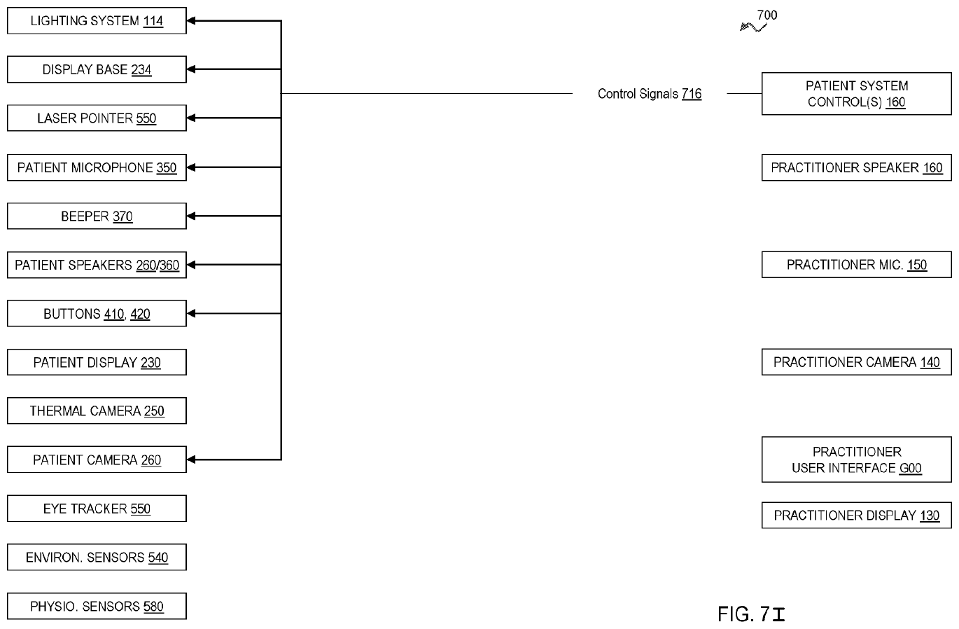

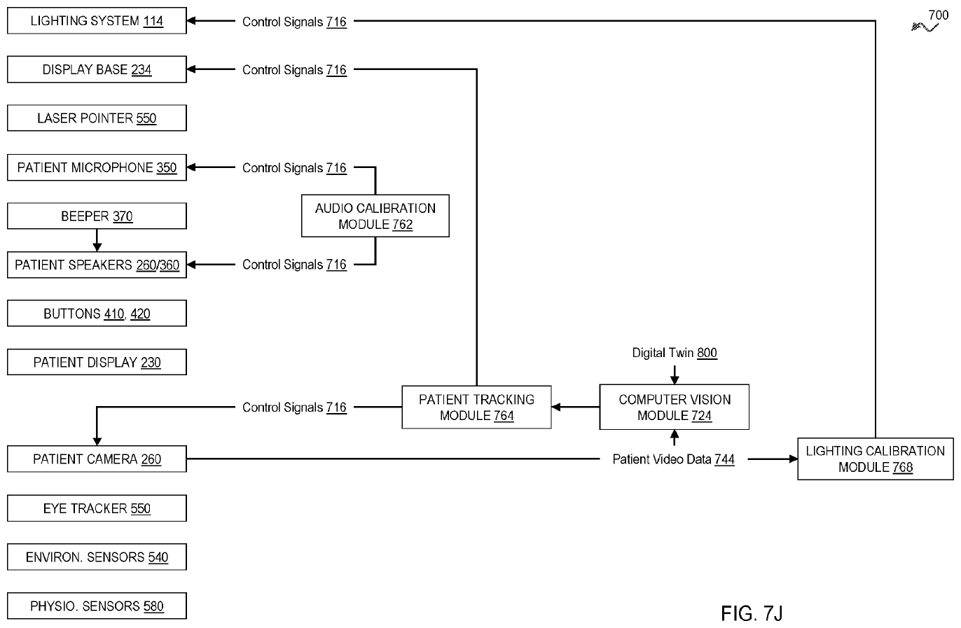

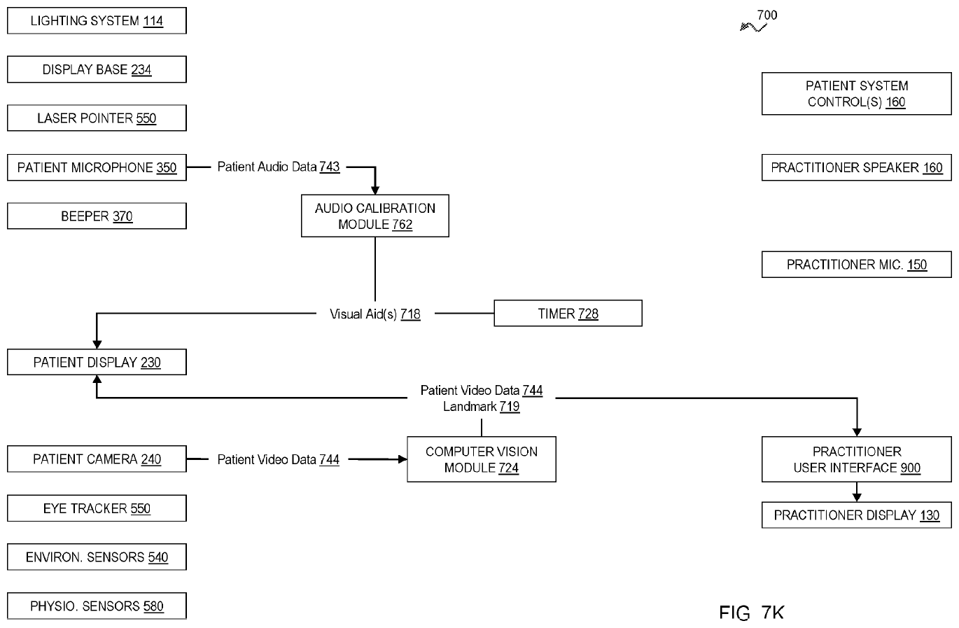

FIGS. 7A through 7K are a block diagrams of some of the software modules 700 and data flow of the cyber-physical system 100 according to exemplary embodiments.

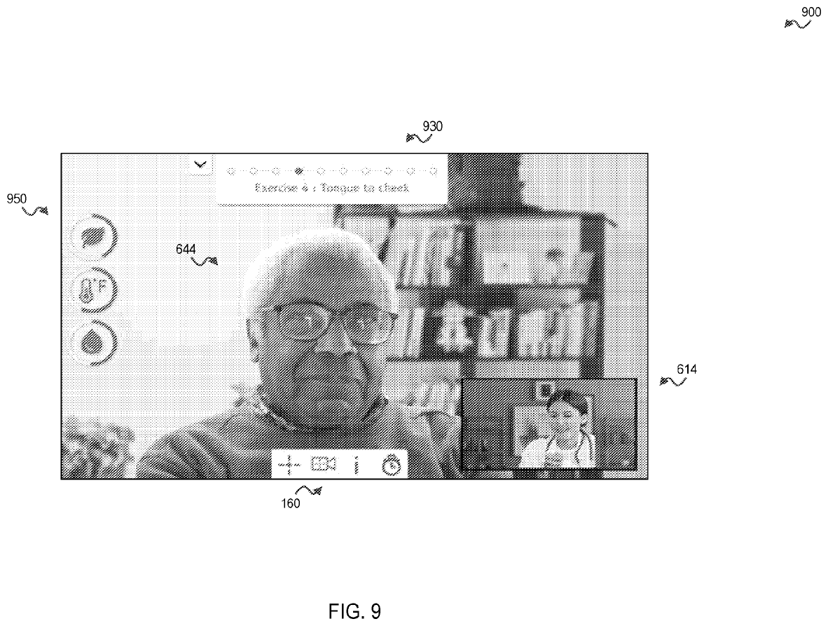

In the embodiment of FIG. 7A, the cyber-physical system 100 includes a videoconferencing module 710, which may be realized as software instructions executed by both the patient computing system 500 and the practitioner system 120. As described above, patient audio data 743 is captured by the patient microphone 350, practitioner audio data 715 is captured by the practitioner microphone 150, practitioner video data 714 is captured by the practitioner camera 140, and patient video data 744 is captured by the patient camera 240. Similar to commercially-available videoconferencing software (e.g., Zoom), the videoconferencing module 710 outputs the patient audio data 743 via the practitioner speaker 160, outputs practitioner audio data 715 via the patient speaker(s) 260 or 360, outputs practitioner video data 714 captured by the practitioner camera 140 via the patient display 230, and outputs patient video data 744 via a practitioner user interface 900 (described in detail below with reference to FIG. 9) on the practitioner display 130.

In order to perform the computer vision analysis described below (e.g., by the patient computing system 500), the patient video data 744 may be captured and/or analyzed at a higher resolution (and/or a higher frame rate, etc.) than is typically used for commercial video conferencing. Similarly, to perform the audio analysis described below; the patient audio data 743 may be captured and/or analyzed at a higher sampling rate, with a larger bit depth, etc., than is typical for commercial video conferencing software. Accordingly, while the patient video data 744 and the patient audio data 743 transmitted to the practitioner system 120 via the communications networks 170 may be compressed, the computer vision and audio analysis described below may be performed (e.g., by the patient computing system 500) using the uncompressed patient video data 744 and/or patient audio data 743.

In the embodiment of FIG. 7B, the cyber-physical system 100 includes a sensor data classification module 720, which includes an audio analysis module 723, a computer vision module 724, a signal analysis module 725, and a timer 728. The sensor data classification module 720 generates physical state variables 820 indicative of the physical state of the patient 101, emotive state variables 840 indicative of the emotive state of the patient 101, cognitive state variables 820 indicative of the cognitive state of the patient 101, and/or social state variables 820 indicative of the social state of the patient 101 (collectively referred to herein as state variables 810) using the patient audio data 743 is captured by the patient microphone 350, the patient video data 744 captured by the patient camera 240, patient responses 741 captured using the buttons 410 and 420, thermal images 742 captured by the thermal camera 250, eye tracking data 745 captured by the eye tracker 550, environmental data 747 captured by one or more environmental sensors 540, and/or physiological data 748 captured by one or more physiological sensors 580 (collectively referred to herein as sensor data 740).

More specifically, the sensor data classification module 720 may be configured to reduce or eliminate noise in the sensor data 740 and perform lower level artificial intelligence algorithms to identify specific patterns in the sensor data 740 and/or classify the sensor data 740 (e.g., as belonging to one of a number of predetermined ranges). In the embodiments of FIGS. 7B through 7K described in detail below, for example, the computer vision module 724 is configured to perform computer vision analysis of the patient video data 744, the audio analysis module 723 is configured to perform audio analysis of the patient audio data 743, and the signal analysis module 725 is configured to perform classical signal analysis of the other sensor data 740 (e.g., the thermal images 742, the eye tracking data 745, the physiological data 748, and/or the environmental data 747).

As described in more detail below with reference to FIGS. 8A-8F, the state variables 810 calculated by the sensor data classification module 720 form a digital twin 800 that may be the input of a heuristic computer reasoning engine 890. Additionally, as described in more detail below with reference to FIG. 9, the sensor data 740 and/or state variables 810 and recommendations from the digital twin 800 and/the heuristic reasoning engine 890 may be displayed to the practitioner 102 via the practitioner user interface 900.

In a clinical setting, for instance, the signal analysis module 725 may identify physical state variables 820 indicative of the physiological condition of the patient 101 (e.g., body temperature, pulse oxygenation, blood pressure, heart rate, etc.) based on physiological data 748 received from one or more physiological sensors 580 (e.g., a thermometer, a pulse oximeter, a blood pressure monitor, an electrocardiogram, data transferred from a wearable health monitor, etc.). Additionally, to provide functionality to identify physical state variables 820 in settings where physiological sensors 580 would be inconvenient or are unavailable, the sensor data classification module 720 may be configured to directly or indirectly identify physical state variables 820 in a non-invasive manner by performing computer vision and/or signal processing using other sensor data 740. For example, the thermal images 742 may be used to track heart beats² and/or measure breathing rates.³

In some embodiments, for instance, the cyber-physical system 100 may be configured to enable the practitioner 102 to conduct a computer-assisted cognitive impairment test of the patient 101,4 such the Automated Neuropsychological Assessment Metrics (ANAM) adapted to elderly people, the Cambridge Neuropsychological Test Automated Battery (CANTAB), MindPulse: Attention and Excecution/Inhibition/Feedback to Difficulty, NeuroTrax, etc. To conduct the cognitive impairment test, test questions may be displayed to the patient 101 via the patient display 230. Meanwhile, because cognitive impairment tests require only yes or no answers, the cyber-physical system 100 enables the patient 101 to easily answer those test questions using the buttons 410 and 420 of the control box 300.

In addition to recording the test questions and the patient responses 741 to those questions, the sensor data classification module 720 uses the timer 728 to record time stamps indicative of when each test question was displayed to the patient display 230 and when each patient response 741 was provided. That time series is typically the only data produced when conducting a typical cognitive impairment test. However, the cyber-physical system 100 may be configured to use a number of input channels to provide a larger spectrum of information useful to the analysis and interpretation of the cognitive impairment test results. In some of those embodiments, for instance, physiological sensors 580 may be used to identify the physiological condition of the patient 101 (for instance, a breath sensor 340 may record physiological data 748 related to alcohol consumption or digestive issues). Additionally, the thermal images 742 may provide the input to one or more algorithms that identify indicators of stress, pain, cognitive load, and potentially vital signs.⁵ Similarly, the eye tracking data 745 may be used to identify evidence of the behavior and level of attention of the patient.⁶ Additionally or alternatively, the computer vision module 724 may analyze the patient video data 744 and use various algorithms to classify facial expressions⁷ and body language⁸ (e.g., to support an interpretation of neurologic condition. Finally, the audio analysis module 723 may perform a multispectral analysis of the patient audio data 743, for example to detect stress and/or deception.⁹

In some embodiments, the cyber-physical system 100 may enable the practitioner 102 to conduct a neurological examination of the patient 101. In those embodiments, the sensor data classification module 720 may be configured to compute the Myasthenia Gravis (MG) core examination metrics, for example by using the computer vision module 724 to identify and track facial and body movements of the patient 101 in the patient video data 744 and/or using the audio analysis module 723 to analyze the patient audio data 743 as outlined below and described in the inventors' forthcoming paper.

During the neurological examination, for example, the practitioner 102 may ask the patient 101 to perform an arm strength exercise and another exercise where the patient need pass from a standing position to seated position. In those instances, the computer vision module 724 may identify and track the movement of body landmarks 701 (e.g., as shown in FIG. 7C) in the patient video data 744 to determine if the patient 101 can perform an arm strength exercise within certain predetermined time periods (e.g., in less than 9 seconds, within 10 to 89 seconds, within 90 to 119 seconds, or more than 120 seconds) and if the patient 101 has difficulty standing from a seating position (e.g., if the patient 101 is unable to stand unassisted, if the patient 101 needs to use his or her hands, or if the patient 101 is slow to rise but does not need to use his or her hands). To identify and track body landmarks 701, for example, the computer vision module 724 may use BlazePose GHUM 3D from MediaPipe.¹⁰

Similarly, the practitioner 102 may ask the patient 101 to perform a cheek puff exercise and a tongue-to-cheek exercise. In those instances, the computer vision module 724 may identify the face and/or eyes of the patient 101 in the patient video data 744 and identify and track face and/or face landmarks 702 (e.g., as shown in FIG. 7D) to determine if the patient 101 can perform those exercises. Additionally, the computer vision module 724 may track the movement of those face and/or eye landmarks 702 to determine if the patient 101 experiences ptosis (eyelid droop) or diplopia double vision within certain predetermined time periods (e.g., in less than 1 second, within 1 to 10 seconds, or within 11 to 45 seconds). To identify and track face landmarks 702, the computer vision module 724 may use any of a number of commonly used algorithms,¹¹ such as the OpenCV implementation of the Haar Cascade algorithm,¹² which is based on the detector developed by Rainer Lienhart.¹³

To assess diplopia, the computer vision module 724 may track eye motion to verify the quality of the exercise, identify the duration of each phase, and register the time stamp of the patient expressing the moment double vision occurs.¹⁴

To assess ptosis as shown in FIG. 7E, for example, deep learning¹⁵ may be used to identify regions of interest 703 in the patient video data 744, identify face landmarks 702 in those regions of interest 703, and measure eye dimension metrics 704 used in the eye motion assessment, such as the distance 705 between upper and lower eye lid, the area 706 of the eye opening, and the distance 707 from the upper lid to the center of the pupil.¹⁵ Because the accuracy of the face landmarks 702 may not be adequate to provide accurate enough eye dimension metrics 704 to assess ptosis and ocular motility,¹⁶ however, the cyber-physical system 100 may superimpose the face landmarks 702 and eye dimension metrics 704 identified using deep learning approach over the regions of interest 703 in the patient video data 744 and provide functionality (e.g., via the practitioner user interface 900) to adjust those distances 705 and 707 and area 706 measurements (e.g., after the neurological examination).

To determine whether the patient 101 can perform the cheek puff exercise, sensor data classification module 720 identifies cheek deformation by measuring the polygon 708 delimited by points (3), (15), (13), and (5) of FIG. 7D. To determine whether the patient 101 can perform the tongue-to-cheek exercise, the region of interest can be restricted to the half of the polygon 708 in which the patient 101 is attempting to press his or her tongue to his or her cheek.

In some embodiments, the cyber-physical system 100 may include a depth camera. In those embodiments, the sensor data classification module 720 may use the three-dimensional image data of the patient 101 to identify the local curvature of the cheek. 17 However, lower cost depth cameras that use infrared and/or stereo images to measure depth (e.g., the Intel Realsense D435) may not be accurate enough to measure cheek deformation (particularly for patients 101 who have difficulty pushing their cheek with their tongues). Meanwhile, more accurate depth cameras that use the time of flight technology to measure depth¹⁸ may be too expensive to include in many embodiments. Accordingly, in most embodiments, the computer vision module 724 uses the patient video data 744 to track mouth deformation and/or change in illumination of the cheek to measure cheek deformation and reproducibility.

Similar to what a medical doctor grades during a telehealth consultation, for example, the computer vision module 724 may determine when the cheek deformation starts, when the cheek deformation ends, and if the cheek deformation gets weaker in time during the examination. For instance, because the local skin appearance changes as it gets dilated,¹⁹ the computer vision module 724 may identify and track a region of interest 703 between the mouth location and the external boundary of the cheek (where supposedly the deformation should be significant) and calculates the average pixel value of the blue dimension of the RGB code over time during the exercise. Additionally or alternatively, because the average pixel value method may depend on skin color and may not be sufficient (e.g., in certain lighting conditions), the computer vision module 724 may take advantage of the fact that cheek deformation impacts mouth geometry and identify cheek puffs and by tracking the movement of facial landmarks 702. For example, the computer vision module 724 may identify a cheek puff by determining whether the lips of the patient 101 take on a more rounded shape. Similarly, the computer vision module 724 may identify a tongue-to-cheek push by determining whether the upper lip is deformed.

During the arm strength assessment, the computer vision module 724 may track movement of the upper body motion patient 101 and a steady position of both arms using a standard deep learning technique,²⁰ which provides a precise metric of the angle of the arm versus body, stability of the horizontal arm position, and duration.

Additionally, the practitioner 102 may ask the patient 101 to count to 50 and count to the highest number possible in a single breath. In those instances, the sensor data classification module 720 may determine the highest number the patient 101 can count to in a single breath (e.g., less than 20, between 20 and 24, between 25 and 29, or more than 30), for example, using a speech recognition algorithm, and/or whether the patient 101 experiences shortness of breath (e.g., shortness of breath with exertion, shortness of breath at rest, or ventilator dependence). In some embodiments, the sensor data classification module 720 may determine whether the patient 101 experiences shortness of breath using a three-dimensional images of the patent 102 captured by a depth camera and/or by analyzing the thermal images to track the extent of the plume of warm air that is exhaled by the patient 101.21 Additionally or alternatively, the audio analysis module 723 may extract features from the patient audio data 743 indicative of shortness of breath, such as:
Loudness of Voice (LV) computed, for example, based on the algorithms defined in ITU-R BS.1770-4 and EBU R 128 standards and integrated over all speech segments.
Pitch or Fundamental Frequency (PFF) of Voice, for example computed for each speech segment and compared to the PFF of a typical adult male (i.e., from 85 to 155 Hz) of the PFF of a typical adult female (i.e., from 165 to 255 Hz).
Spectral energy on a frequency interval, for example by computing the L2 norm spectral energy of the voice signal over all speech segment in a frequency window (e.g., between 5 Hz and 25 Hz) that focus on breathing rate.
The Teager and Kaiser energy that is used in tone detection.²²
The Spectral Entropy (SE) of the voice signal, for example by treating the voice signals normalized power distribution in the frequency domain as a probability distribution and calculating the Shannon entropy of the normalized power distribution. Shannon entropy has been used for feature extraction in fault detection and diagnosis.²³ Spectral entropy is also widely used as a feature in speech recognition²⁴ and biomedical signal processing.²⁵
Special feature of the single breath count. Because the airflow volume expanded during speech is in first approximation related to the square of the amplitude of the sound wave, the audio analysis module 723 may compute the integral of the square of the amplitude of the sound wave during the time window the patient speech. In embodiments where the patient microphone 350 is not calibrated or there is a lot of variability on diction during this exercise (e.g., some or patients 101 take their time to count while others pronounce numbers very quickly), the audio analysis module 723 may also compute the percentage of time with vocal sound versus total time as an additional feature.

In addition to the standard MG scores described above, the sensor data classification module 720 may also be configured to calculate state variables 810 indicative of the dynamic of neuromuscular weakness of the patient 101 during the time interval of each exercise, which are essential to create a digital twin 800 of neuromuscular weakness. For instance, an essential model can assimilate the core examination data for each of the following muscles groups: left and right eyes, tongue to left cheek and right cheek, pulmonary diaphragm, left arm and right arm, left leg and right leg. Because each fatigue exercise corresponds to the activation by the central nervous system of one of those muscle groups for a specific duration, the sensor data classification module 720 may calculate a time dependent curve that represents the physical response of that activation. A simple three compartment model of muscle fatigue can be expressed as follows:

$$\frac{{dM}_{A}}{dt} = {BM}_{uc} - {FM}_{A}$$

$$\frac{{dM}_{F}}{dt} = {FM}_{A} - {RM}_{F}$$

$$\frac{{dM}_{uc}}{dt} = {RM}_{F} - {BM}_{uc}$$

$${M}_{A} + {M}_{F} + {M}_{uc} = {M}_{0}$$

where t corresponds to the time scale of the physical exercise, M₀ is the total available motor units (decomposed into the group of activated muscles M_{A}, already fatigued muscles M_{F} and muscles at rest M_{uc}), B is the activation rate, F is the fatigue rate, and R is the recovery rate. The model of muscle fatigue above is inspired by Jing et al.,²⁶ except that loop cycling is used between the three compartments. Additionally, while there is always a residual positive amount of muscle activated in the model of Jing et al., the model of muscle fatigue above leads to a limit state that is zero for the available motor units of muscles at rest M_{uc}, which seems more realistic from the biological point of view. Because the system of differential equations is linear with constant coefficients (i.e., the activation rate B, the fatigue rate F, and the recovery rate R), it is straightforward to write down the explicit solution and check the asymptotic behavior as in Jing et al.

The model of muscle fatigue may be modified to take into account the potential defective activation of the muscle fibers due to a generic auto-immune factor. For example, an autoimmune response Q may be modulated in each muscles group by a generic vascularization factor that takes into account the local distribution of the autoimmune factor. The model may be stochastic, meaning the activation comes with a probability of activation components p(N), where N is the order of magnitude of the number of muscle fibers. For instance, a phenomenological model of muscle fatigue for each muscle group having an index j may be expressed as follows:

$$\frac{{dM}_{A}^{j}}{dt} = \frac{{B}_{j}}{{Q}_{j}} {M}_{uc}^{j} - {F}_{j} {M}_{A}^{j}$$

$$\frac{{dM}_{F}^{j}}{dt} = {F}_{j} {M}_{A}^{j} - {R}_{j} {M}_{F}^{j}$$

$$\frac{{dM}_{uc}^{j}}{dt} = {R}_{j} {M}_{F}^{j} - \frac{{B}_{j}}{{Q}_{j}} {M}_{uc}^{j}$$

$${Q}_{j} = {Q}^{0} {V}_{j} p ( {N}_{j} )$$

where Q⁰ is a generic autoimmune factor that is common to all muscle groups and, for each muscle group having an index j, Qⱼ is the autoimmune factor, Vⱼ represents the impact of vascularization on that muscle group, Nⱼ is the number of muscle fibers, M_{A}^{j} is the available motor units of activated muscles, M_{F}^{j} is the available motor units of already fatigued muscles, M_{uc}^{j} is the available motor units of muscles at rest, Bⱼ is the activation rate, Fⱼ is the fatigue rate, Rⱼ is the recovery rate, M_{A}^{j}+M_{F}^{j}+M_{uc}^{j}=1 after normalization, and p(Nⱼ) is a probability distribution that gets close to a bell function as the number of fibers Nⱼ→∞. The phenomenological model above is designed in such a way that:
The baseline autoimmune factor Q⁰, a characteristic component of the disease, is common to all muscle groups.
The larger the autoimmune factor Q, the less activation there is.
The larger the vascularization V, the larger the autoimmune response.
The larger the number of fibers Nⱼ in the muscle group, the less stochastic variation one can expect.

FIG. 7F illustrates a stochastic run of the phenomenological model described above. By fitting the phenomenological model to the response curves for each muscle group obtained from the patient video data 744 by the computer vision module 724 (for example, as shown in FIGS. 7G and 7H), the sensor data classification module 720 can recover the unknown parameters Q₀ and Bⱼ, Fⱼ, Rⱼ for each muscle group.

The digitalization of state variables 810 indicative of the dynamic of neuromuscular weakness of the patient 101 enables the system 100 (e.g., the server 180) to build a large unbiased database of 182 the patient 101. The quality of the dataset supports the classification of the treatment of patients 101 as a function of the severity of the score in each of the above categories, as well as the fitting of a stochastic dynamic system:

$$\frac{d}{dT} \vec{S} ( T ) = F ( \vec{S} ( T ) , \vec{C} ( T ) )$$

where \vec{S}(T) is the state variable 810 describing the MG patient condition and \vec{C}(T) is the control variable corresponding to drug treatment, and T is the long-time scale of the patient disease (as opposed to the short time scale t of the core physical examination). Overall, the digital twin 800 is then multiscale in time. To be more specific, the vector S(T) contains at minimum the baseline autoimmune Q⁰ that is common to all muscle groups and may include gene regulation factors (as in the model of vascular adaptation described in Casarin et al.²⁷). The control variable \vec{C}(T) may focus on the drug treatment and have comorbidity factors as well.

The cyber-physical system 100 provides unique advantages compared to traditional telehealth systems. Referring back briefly to FIGS. 3 and 4, for instance, the control box 300 of may enable the patient 101 to establish a telehealth session with the simple click of a button 410 or 420 on the top of a control box 300 that is connected (e.g., wirelessly) to the telehealth system on the patient side. Alternatively, the system 100 may enable the patient 101 to establish a telehealth session by saying a simple voice command by use voice recognition to identify a command to establish the telehealth session and/or disconnect the communication. In some embodiments, the system 100 has no key board, no mouse, and no cumbersome cables—just one control box 300 with two obvious control buttons 410 and 420 that light up as needed, for example to participate in the cognitive impairment test described above. The control box 300 may also be equipped with a beeper 350 to help the patient 101 find the box, which may be particularly useful for patients 101 with cognitive impairment and memory loss as described above.

As shown in FIG. 7I, the cyber-physical system 100 provides patient system controls 160, enabling the practitioner 102 to output control signals 716 to control the pan, tilt, and/or zoom of the patient camera 260, adjust the volume of the patient speakers 260 and/or the sensitivity of the patient microphone 350, activate the beeper 370 and/or illumine the buttons 410 and 420 to help the patient 101 find the control box 300 as described above, activate and control the direction of the laser pointer 550, rotate and/or tilt the display base 234, and/or adjust the brightness of the lighting system 114. The patient system controls 160 may be, for example, a hardware device or a software program provided by the practitioner system 120 and executable using the practitioner user interface 900.

Accordingly, once the telehealth connection is established, the cyber-physical system 100 enables the practitioner 102 to get the best view of the patient 101, zoom in and zoom out in the regions of interest 703 important to the diagnosis, orient the patient display 230 so the patient 101 is well positioned to view the practitioner 102, and control the sound volume of the patient speaker 260 and/or 360, the sensitivity of the patient microphone 350, and the brightness of the lighting in the patient environment 110. Accordingly, the practitioner 102 benefits from a much better view of the region of interest than with an ordinary telehealth system. For example, it would be much more difficult to ask an elderly patient 101 to hold a camera toward the region of interest to get the same quality of view.

As shown in FIG. 7J, control signals 716 may also be output by an audio calibration module 762, a patient tracking module 764, and/or a lighting calibration module 768.

Traditional telemedicine systems can introduce significant variability in the data acquisition process (e.g., patient audio data 743 recorded at an inconsistent volume, patent video data 744 recorded in inconsistent lighting conditions). In order to calculate accurate state variables 810, it is important to reduce that variability, particularly when capturing sensor data 740 from the same patient 101 over multiple telehealth sessions. Accordingly, the cyber-physical system 100 may output control signals 716 to reduce variability in the data acquisition process. For example, the lighting calibration module 768 may determine the brightness of the patient video data 744 and output control signals 716 to the lighting system 114 to adjust the brightness in the patient environment 110.

As described above, the control box 300 may include a microphone 350 in order to capture better the voice of the patient 101. Additionally, the audio calibration module 762 may form a feedback loop to calibrate the sound volume of the patient speaker 360 and/or the sensitivity of the patient microphone 350. For example, the beeper 370 may output a consistent tone (e.g., via the patient speaker 360), which may be captured by audio calibration module 762 via the patient microphone 350. The audio calibration module 762 may then calculate the volume (for example, using algorithms defined in ITU-R BS.1770-4 and EBU R 128 standards) and adjust the sound volume of the patient speaker 360 and/or the sensitivity of the patient microphone 350.

The patient tracking module 764 may use the patient video data 744 to track the location of the patient 101 and output control signals 716 to the patient camera 260 (to capture images of the patient 101) and/or to the display base 234 to rotate and/or tilt the patient display 230 towards the patient 101. Additionally or alternatively, the patient tracking module 764 may adjust the pan, tilt, and/or zoom of the patient camera 260 to automatically provide a view selected by the practitioner 102 (e.g., centered on the face of the patient 101, capturing the upper body of the patient 101, a view for a dialogue with the patient 101 and a nurse or family member, etc.), or to provide a focused view of interest based on sensor interpretation of vital signs or body language in autopilot mode.²⁸

In preferred embodiments, the patient tracking module 764 automatically adjusts the pan, tilt, and/or zoom of the patient camera 260 to capture each region of interest 703 relevant to each assessment being performed. As shown in FIG. 7J, for instance, the computer vision module 724 identifies the regions of interest 703 in the patient video data 744 and the patient tracking module 764 outputs control signals 716 to the patient camera 260 to zoom in on the relevant region of interest 703. Generic artificial intelligence and computer vision algorithms may be insufficient identify the specific body parts of patients 101, particularly patients 101 having certain conditions (such as Myasthenia Gravis). However, the cyber-physical system 100 has access to the digital twin 800 of the patient 101, which includes a mathematical representation of biological characteristics of the patent 101 (e.g., eye color, height, weight, distances between body landmarks 701 and face landmarks 702, etc.). Therefore, the digital twin 800 may be provided to the computer vision module 724. Accordingly, the computer vision module 724 is able to use that specific knowledge of the patient 101—together with general artificial intelligence and computer vision algorithms—to identify the regions of interest 703 in the patient video data 744 so that the patient camera 260 can zoom in on the region of interest 703 that relevant to the particular assessment being performed.

Additionally, to the limit any undesired impact on the emotional and social state of the patient 101 caused by the telehealth session, in some embodiments the cyber-physical system 100 may monitor the emotive state variables 840 and/or social state variables 880 of the patient 101 and, in response to changes in the emotive state variables 840 and/or social state variables 880 of the patient 101, adjust the view output by the patient display 230, the sounds output via the patient speakers 260 and/or 360, and or the lights output by the lighting system 114 and/or the buttons 410 and 420 (e.g., according to preferences specified by the practitioner 102) to minimize those changes in the emotive state variables 840 and/or social state variables 880 of the patient 101.

As shown in FIG. 7K, the cyber-physical system 100 may also output visual aids 718 to assist the patient 101 and/or the practitioner 102 to capture sensor data 720 using a consistent process.

In the counting to 50 and single breath counting exercises described above, for example, the patient 101 may output different airflow depending on how quickly and loudly the patient 101 is counting. Accordingly, the timer 728 may be used to provide a visual aid 718 (e.g., via the patient display 230) to guide the patient 101 to count with a consistent rhythm of about one number counted per second. Additionally, to ensure that patient audio data 743 is captured at a consistent volume as described above, the audio calibration module 762 may analyze the patient audio data 743 and provide a visual aid 718 to the patient 101 (e.g., in real time) instructing the patient 101 to speak at a higher or lower volume.

Additionally, digitalization of the ptosis, diplopia, cheek puff, tongue-to-cheek, arm strength, and stand-to-sit assessments depends heavily on controlling the framing of the regions of interest 703 (and the distance from the camera patient camera 240 to the region of interest 703). Therefore, the patient video data 744 may be output to the patient 101 (and/or the practitioner 102) with a landmark 719 (e.g., a silhouette showing the desired size of the patient 101) so the practitioner 102 can make sure the patient 101 is properly centered and distanced from the patient camera 240).

Additionally, in some embodiments, the cyber-physical system 100 provides the practitioner 102 with real-time environmental data 747 indicative of the environmental conditions (e.g., temperature, humidity, and airborne particle rates) in the patient environment 110 so that the practitioner 102 may assess the parameters that may affect the health conditions of the patient.²⁹ For example, senior patients often do not realize that they get dehydrated if the temperature is high and the humidity is low or that they risk pneumonia if the temperature is low and the humidity is high. Similarly, high particle counts in the air due to a lack of room ventilation are related to a greater risk of airborne disease. All those factors, for instance, may contribute to an abnormally low cognitive performance. (The environmental data 747 may be used as inputs of the digital twin 800 of the patient 101 as a controlled variables because they impact the evolution of the principle state variables 810.) As described below with reference to FIG. 9, for example, the practitioner user interface 900 may superimpose the environmental data 747 in the video view of the patient 101, on demand. With the addition of the environmental data 747, the practitioner 102 can provide improved medical diagnostics that are even more precise than the ones possible during a consultation at the doctor's office.

The cyber-physical system 100 even provides features to address the main draw back of telehealth consultations: the inability of practitioners 102 to physically interact with the patient 101. Examining a patient 191 complaining of abdominal pain, for instance, the thermal imaging camera 250 may detect inflammation that manifests by an intense sub-surface vascularization flow.³⁰ Because the practitioner 102 cannot touch the patient 101 to localize pain points and abdomen stiffness, a practitioner 102 using a traditional telemedicine platform is typically reduced to asking the patient 111 to press on their abdomen at specific locations and get feedback on pain or discomfort. However, cyber-physical system 100 offers possibilities to mitigate that difficulty. First, a laser pointer 550 may be mounted on top of the patient display 230, which can be activated to show the patient precisely where the doctor's region of interest is located. The patient camera 240 system can automatically track the region of interest by using either the laser pointer 250 landmark or a thermal map of the abdomen generated using the thermal images 742. Second, using the computer vision module 724 and the thermal images 742, the practitioner 102 can get an indirect assessment of abdominal stiffness and local inflammation. Finally, as mentioned above, the pain level can be analyzed from facial expressions and correlated to the patient feedback registered in the doctor's medical notes.

In some embodiments, the cyber-physical system 100 can also be used in conjunction with a patient surveillance system and quickly and automatically establish telehealth communication with medical staff when needed. For example, a patient 101 (e.g., having Alzheimer's disease) in the clinical setting of FIG. 2B may fall from their bed 201 onto the floor. A patient surveillance system³¹ may detect that the patient is no longer on their bed. If the patient's presence on the bed is not detected for a preset period of time, for example, the cyber-physical system 100 may wake up and activate the thermal imaging camera 250. A fast computer vision algorithm of the thermal image 742 may detect the heat of the body that is lying on the floor. The thermal image analysis is not intrusive in the infrared spectrum and can be easily interpreted.³² Based on these two concurring signals (i.e., no patient 101 on the bed 201 and the patient 101 detected on the floor), the cyber physical system 100 can automatically start running the heuristic computer reasoning system 890 described below; relying on the digital twin 800 of the patient 101 described below; to decide whether or not to go into telehealth mode and establish the connection with the medical staff on call to send the relevant information. Medical staff can them quickly try to communicate with the patient 101 and run a quick visual assessment with the remotely-controllable camera 240. Then, if it is decided to send someone to help, the provider may use the system 100 to provide comfort and reassurance that help is on the way.

In each utilization scenario described above, the input provided to the system is rich enough to allow the construction of a digital twin 800 of the patient, the same way it can be with a genome,³³ but with a scalable database that integrates multiple modalities related to anatomy, behavior, and environmental conditions of the patients 101.

FIG. 8A illustrates the construction of a digital twin 800 of the patient 101 according to an exemplary embodiment.

In principle, a digital twin is “a virtual representation that serves as the real-time digital counterpart of a physical object or process” and was first introduced for manufacturing processes.³⁴ A digital twin 800 of patient 101 may be far more complex and may require a strong ethical principal.³⁵ In the cyber-physical system 100 described herein, the digital twin 800 is a model that can behave as a user of a telehealth system. The digital twin 800 of the patient 101 is an agent-based model with state variables 810 that represent the anatomic and metabolic variables of the patient 101 as well as a behavior state related to stress, cognitive load, memory components, etc. Statistical rules describe how the dynamical system transitions from one set of state variable values 810 to another in time. State variables 810 are preferably discrete numbers, and statistical rules are parametrized. Such an agent-based model has hundreds of unknown parameters that can be interpreted as the “gene set” of the user with respect to the model (similar to the inventors' work in system biology³⁶). As described below, those unknown parameters are calibrated using telehealth data. Meanwhile, the model is calibrated dynamically and keeps evolving with the patient observations accumulated at each telehealth session.

As shown in FIG. 8A, all present and past input of the telehealth session (e.g., patient audio data 743, patient video data 744, patient responses 741, thermal images 742, eyetracking data 745, environmental data 747, physiological data 748, notes from the practitioner 102) are input into the digital twin 800 of the patient 101.

The digital twin 800 of the patient 101 is specific to the disease management and describes the dynamic of the variables (e.g., physical state variables 820, emotive state variables 840, cognitive state variables 860, and/or social state variables 880) under the stimuli of the cognitive test or medical examination run by the practitioner 102.

The physical state variables 820 are models specific to the disease. In the Myasthenia Gravis (MG) example above, for instance, the physical state variables 820 may include eye motion, upper body motion, facial motion focusing on lip when talking, or cheek when the patient uses their tongue. As described above, the sensor data classification module 720 may directly or indirectly recover many physical state variables 820 through the sensor data 740 using computer vision and signal processing. As described below with reference to FIG. 8B, the digital twin 800 operates on those physical state variables 820 with a stochastic operator 899 and can damp the noise on measurement to some extent.

The emotive state variables 840 may include indicators on happiness, sadness, fear, surprise, disgust, anger or being neutral. As described above, the sensor data classification module 720 may identify many emotive state variables 840 through facial expression and eye tracking.

The cognitive state variables 860 include the ability to process a specific task and/or the ability to memorize specific data. Typically, the telehealth session concentrates on a specific cognitive variable to establish a diagnostic and monitor the progression of a disease.

As described above, the cyber-physical system 100 may be used to conduct a cognitive assessment and the sensor data classification module 720 may identify many cognitive state variables 860 through sensor data 740. As such, the cyber-physical system 100 can be used in controlled consultations (such as the consultations for Myasthenia Gravis patients 101 described above) and can also be beneficial on routine consultations for a patient 101 without chronic disease.

The social state variables 880 measure some aspect of aggressive behavior, mutualistic behavior, cooperative behavior, altruistic behavior, and parental behavior.

Emotive state variables 840, cognitive state variables 860, and social state variables 880 are correlated with noninvasive measurements during human computer interaction³⁷ and may be identified using the sensor data 740 captured by the sensor array of the cyber-physical system 100.³⁸

The digital twin 800 can be used also to support heuristic computer reasoning engine 890 to deliver telehealth assistance.

Based on the digital twin 800, the heuristic computer reasoning engine 890 runs an artificial intelligence algorithm (on-site or in the cloud) that can reconciliate all these data sets and produce a high-quality analytical report that supports the provider's diagnostic analysis and decisions. The heuristic computer reasoning engine 890 continually consults the digital twin 800 of the patient 101 to assist the practitioner 102 with the workflow process—starting with the medical history and updates and following with the analytical processing of cognitive and other test results in the context of all of the sensor data 740 described above—to support a high quality clinical practice.

The heuristic computer reasoning engine 890 may be, for example, the HERALD system described in PCT Pub. No. 2022/217263, which describes a broad spectrum of applications that have been tested in clinical conditions to improve workflow efficiency and safety and is hereby incorporated by reference. The HERALD system 890 provides the architecture of a heuristic computer reasoning system that assists in the optimized efficiency of workflow by exercising the digital process analogue of a “thought experiment.” The heuristic computer reasoning engine 890 is comprised of a “learning” algorithm, developed as a system. For example, HERALD 890 can assist a system operator in charge of a medical facility with complex workflow efficiency issues and multiple human factor contingencies to gain a higher level of certainty in processes and increased efficiency in resources utilization-both staff and equipment. The same architecture can be a heuristic computer reasoning system 890 that coaches an individual to improve their behavior according to some specific objective such as work quality, quality of health or enhanced productivity.

Once the heuristic computer reasoning system 890 is in place, it allows the construction of a sentient computer system that generates its own most relevant questions and provides optimum decision-making support to an operator based on continuous monitoring of working environments through multiple inputs and sensors. Not only does the present system allow for a more statistically based, informed decision-making tool, but also the system monitors collected data with a continually adaptive system in order to more accurately predict potential future outcomes and events. As more data is collected and as data sets expand, the inherent predictive value of the system increases through enhanced accuracy and reliability. This value is manifested in increasingly efficient workflow operations which may be best reflected through resultant changes in the principal's behavior and overall satisfaction.

The cyber-physical system 100 uses the same three components as the HERALD system, including sensing (set of low-level artificial intelligence algorithms running on sensor output that acquire multimodalities input from the sensor array mounted on the display and patient input), communication (a medium level layer of artificial intelligence to communicate with the medical doctor running the telehealth session either by text messages, graphic user interface or voice recognition), and an evolving model of the problem domain for the patient consultation to support heuristic reasoning using a customized digital twin 800 of the medical doctor workflow that assimilates data from the sensors and communications to best describe the clinical practice.

Once this agent based model is in place, or any dynamical system that will mimic patients' reactions to external stimuli such as environmental conditions, medical conditions including those induced by drugs, questions given by the providers, etc. one can exercise the heuristic computer reasoning schema 890 to support the telehealth session of the provider. As shown in FIG. 8, three categories of actions that the cyber-physical system 100 may provide are: (1) assisting the workflow of the telehealth session to make sure that all steps are covered and there is no gap in the data acquisition that will limit the quality of the diagnostic: (2) providing rational support and analytics on test results and quantified behavior indicators to document the report of the medical doctor as the telehealth session progresses; and (3) providing advanced warning on potential pitfalls based on observations exercising relevant modalities of the sensors to detect when patients start to behave outside “normal parameters” and/or run the HERALD system to constantly check on what might be overlooked with patient conditions.

As shown in FIGS. 8B and 8C, the digital twin 800 of the patient 101 continues to improve by comparing prediction with realization, using for example a mathematical genetic algorithm that optimizes the objective function defined by best clinical practices in the specific medical domain. The objective function might be vastly different in palliative care than in the management of a disease toward recovery. Further, as detailed in PCT Pub. No. 2022/217263, the system must operate within the parameter bounds defined by ethical principles.

Traditionally, clinical decision of provider during a telehealth or in person visit operate on a decision tree.³⁹ This is a standard practice used by provider to determine the best course of action. The simplicity of the decision tree graph makes the construction of the clinical decision algorithm amenable to artificial intelligence techniques such as vector machine [65],⁴⁰ random forest,⁴¹ and deep learning.⁴² The simplicity and ease of use of clinical decision tree make the method very popular. However, decision trees operate on discrete sets, ignoring the continuity in time and state variable space of the patient condition and eventually erasing any nuance in clinical decision. Decision trees also have the tendency to suppress the critical thinking needed in precision medicine and leave the practitioner 102 with little choice under the assumption that the clinical tree decision is standard of care.

By contrast, as shown in FIG. 8C, the digital twin 800 approach to clinical decision support described herein is a continuous, stochastic process that predicts the patient trajectory with respect to his/her state variable 810 with respect to time. In particular, the observed errors between prediction and observation during the follow up of the patient offers an opportunity to retrofit the digital twin 800 to be more specific to the patient. Accordingly, the cyber-physical system 100 automatically computes a score 808 of each step of the medical exam (e.g., of the MG patients described above) and updates those scores 808 in real-time using the digital twin 800 model, providing an unvaluable dataset for use in clinical trials (e.g., to evaluate the effectiveness of drugs used to control neurological disease) and other telemedicine applications.

FIG. 8B is a diagram of the stochastic process used to retrofit the digital twin 800 according to an exemplary embodiment.

As shown in FIG. 8B, each state variable 810 includes a known state variable 801 and a hidden state variable 809. In response to each observation made by the cyber-physical system 100 or answer provided by the patient 101, the digital twin 800 is retrofit to reduce the amount of the hidden variable 809 improve the predictability of the state variable 810. A projection operator is used to translate the state variable 810 into an objective score 808 to assess a disease (e.g., a Myasthenia Gravis core examination metric).

The digital twin 800 provides a rigorous mathematical framework that allows the heuristic computer reasoning engine 890 to use artificial intelligence to systematically test out clinical options by generating and simulating “what if?” considerations. As shown in FIG. 8D, for example, the heuristic computer reasoning engine 890 can perform an exploration forward in time by simulating a change in a state variable 810. Similarly, shown in FIG. 8E, the heuristic computer reasoning engine 890 can simulate a change in a control variable. Meanwhile, as shown in FIG. 8F, the heuristic computer reasoning engine 890 can perform a backward search to analyze the potential of causality in patient conditions.

By reducing the prospective and retrospective analysis to mathematical formulations executed using the digital twin 800 as described below, the heuristic computer reasoning engine 890 can suggest answers to prefactual questions, suggest answers to counterfactual questions, suggest answers to semi-factual questions, suggest answers to predictive questions, perform hind casting, perform retrodiction, perform backcasting, etc.

The following mathematical framework may be used to implement the heuristic computer reasoning engine 890:
\vec{S}(t) is a state variable 810 at time t
\vec{S}(t)+Δ\vec{S}(t) is a new event that updates the state variable 810 at time t
\vec{C}(t) is a control variable at time t
\vec{C}(t)+Δ\vec{C}(t) is a new change on the control variable at time t
An observation [symbol] (Δt) that tracks the state variable evolution in time for a given control variable may be written as:

$$( Δ t ) : ( \vec{S} ( t ) , \vec{C} ( t ) ) \to \vec{S} ( t + Δ t )$$

A simulation algorithm [symbol] (Δt) that predicts the state variable evolution in time for a given control variable may be written as:

$$( Δ t ) : ( \vec{S} ( t ) , \vec{C} ( t ) ) \to \vec{S} ( t + Δ t )$$

Projection of the state into the objective value space is denoted

$$: \vec{S} ( t ) \to \vec{P} ( t )$$

The projected value is a multidimentional vector (P₁, . . . , P_{N}).
The objective function is the weighted norm

$${‖ \vec{P} ‖}_{ω} = ‖ ( {ω}_{1} {P}_{1} , … {ω}_{N} {P}_{N} ‖$$

where ωᵢ≥0, i=1 . . . N.
Measuring the difference between realization and simulation on the objective value can be written as:

$$‖ ℰ ( Δ t ) ( \vec{S} ( t ) , \vec{C} ( t ) ) ‖ = ‖ 𝒫 ( 𝒪 ( Δ t ) ( \vec{S} ( t ) , \vec{C} ( t ) ) ) - 𝒫 ( 𝒮 ( Δ t ) ( \vec{S} ( t ) , \vec{C} ( t ) ) ) ‖$$

The continuity of error estimate with respect to the state variable may be written as:

$$\vert ℰ ( Δ t ) ( \vec{S} ( t ) , \vec{C} ( t ) ) - ℰ ( Δ t ) ( \vec{{S}_{0}} ( t ) , \vec{C} ( t ) ) \vert \leq κ ( {S}_{0} ) ‖ S ( t ) - {S}_{0} ( t ) ‖$$

where κ(S₀) denotes a real number that depends only on the state value S₀.
The continuity of error estimate with respect to the control variable may be written as

$$\vert ℰ ( Δ t ) ( \vec{S} ( t ) , \vec{C} ( t ) ) - ℰ ( Δ t ) ( \vec{S} ( t ) , \vec{{C}_{0}} ( t ) ) \vert \leq χ ( {C}_{0} ) ‖ C ( t ) - {C}_{0} ( t ) ‖$$

where κ(C₀) denotes a real number that depends only on the control value C₀

The heuristic computer reasoning engine 890 can suggest answers to prefactual questions (i.e., “What will be the outcome if event X occurs?”). The abstract formulation of event X is a sudden change of the state variable \vec{S}, denoted Δ\vec{S}. The mathematical formation of this question is

$$( ( Δ t ) ( \vec{S} ( t ) + Δ \vec{S} , \vec{C} ( t ) ) ?$$

The algorithm to answer that problem is based on a forward digital twin run:

$$( ( Δ t ) ( \vec{S} ( t ) + Δ \vec{S} , \vec{C} ( t ) )$$

Accordingly, heuristic computer reasoning engine 890 can suggest answers to prefactual questions, for example:
What if patient is aware of his medical cognitive test results in real time?
What is the impact on Ptosis if the telehealth session is in the late afternoon?

The heuristic computer reasoning engine 890 can suggest answers to counterfactual questions (i.e., “What might have happened if X had happened instead of Y?”). The mathematical formulation of this question is the same as above provided that Δ\vec{S} denotes the difference on the state variable between the sudden change of state variable switching from event X to event Y. Accordingly, the heuristic computer reasoning engine 890 can suggest answers to counterfactual questions, for example:
What would have been the breath assessment if the patient was on drug x instead of drug y?
What if diplopia time had a lag by xx second in the medical exam?
What if the order of question A and B in the cognitive test was reversed?

The heuristic computer reasoning engine 890 can suggest answers to semi-factual questions (i.e., “Even though X occurs instead of Y, would Z still occur?”). For instance, let's assume that event Z corresponds to the j component of the projected value in the objective space. Using Δ\vec{S} defined as above, the mathematical formulation of this question would be:

$${j}_{} ( ( \vec{S} ( t ) + Δ \vec{S} , \vec{C} ( t ) ) - {j}_{} ( ( Δ t ) ( \vec{S} ( t ) , \vec{C} ( t ) ) = 0 ?$$

The heuristic computer reasoning engine 890 answers that question by substituting the digital twin simulation to the observation. Accordingly, the heuristic computer reasoning engine 890 can suggest answers to semi-factual questions, for example:
Even though the answer of question A in cognitive test was incorrect, can question B answered correctly?
Even though ptosis metric x was above y value, can diplopia occur at z angle?

The heuristic computer reasoning engine 890 can suggest answers to predictive questions (i.e., “Can we provide forecasting from stage Z?”). To formulate that question in a more rigorous way, we need to specify how further in time we like this prediction to be, i.e., set ΔT, and how accurate should be this prediction to be valuable. For instance, let's assume \vec{ϵ} to be the tolerance for an admissible prediction value in the objective value space. The mathematical formulation of the question is:

$$∀ j ∈ ( 1 … N ) , \vert {( ℰ ( Δ t ) ( \vec{S} ( t ) , \vec{C} ( t ) ) )}_{j} \vert < \vec{{ϵ}_{J}} ?$$

If this inequality is satisfied the prediction is correct for each component of the objective function. On one end, the heuristic computer reasoning engine 890 checks that inequality comparing observation with simulation and starting from some specific state value in the region of interest. On the other hand, the heuristic computer reasoning engine 890 uses the continuity of the of error estimate with respect to state variable and this past observation to answer that question for any state values closer enough to S(t). Accordingly, the heuristic computer reasoning engine 890 can suggest answers to predictive questions, for example:
Can we predict the evolution of cognitive test x if the average patient's sleep is one hour less?
Can we predict the patient satisfaction rating change if the telehealth sessions are 30 min late on average?
Can we predict end of the clinic day with high confidence at time X of the day?
Can we predict at 7 am which telehealth session will be canceled?),

The heuristic computer reasoning engine 890 can perform hind casting (i.e., “Can we provide forecasting from stage Z with new event X?”). That question is no different than the previous one except it applies to \vec{S}(t)+Δ\vec{S} where Δ\vec{S} stands for new event X:

$$∀ j ∈ ( 1 … N ) , \vert {( ℰ ( Δ t ) ( \vec{S} ( t ) , \vec{C} ( t ) ) )}_{j} \vert < \vec{{ϵ}_{J}} ?$$

The heuristic computer reasoning engine 890 uses the same learning process from past experience described above to handle that problem. Accordingly, the heuristic computer reasoning engine 890 can perform hind casting, for example:
Can we assess how the next telehealth patient sagenda will be delayed since patient X may take Y more minutes?
Patient Y has canceled, how much time of the day may provider z lose or gain?
Can we predict degradation of ptosis metric x, if patient did not take drug y with the correct dosage?

The heuristic computer reasoning engine 890 can perform retrodiction (i.e., past observations, events and data are used as evidence to infer the process(es) that produced them). The heuristic computer reasoning engine 890 starts from a past observation

$$( Δ t ) ( \vec{S} ( t - Δ t ) , \vec{C} ( t - Δ t ) = \vec{S} ( t )$$

that has been tracking the state variable evolution in time for a given control variable. The heuristic computer reasoning engine 890 starts verifies that the model has been predictive:

$$∀ j ∈ ( 1 … N ) , \vert {( ℰ ( Δ t ) ( \vec{S} ( t - Δ t ) ) ( \vec{C} ( t - Δ t ) ) )}_{j} \vert < \vec{{ϵ}_{J}} ?$$

The heuristic computer reasoning engine 890 assumes that the error estimate is continuous with respect to the state variable. The mathematical formulation of retrodiction can be done in many different ways depending on the level of causality the heuristic computer reasoning engine 890 are looking for. In its simplest form, the heuristic computer reasoning engine 890 looks for the variable component that changes the outcome significantly, i.e.:

Find j∈(1 . . . N), such that |(ε(Δt)(\vec{S}(t−Δt)+ΔS(t−Δt),\vec{C}(t−Δt)))ⱼ|>>\vec{∈}ⱼ?

That problem is amenable to standard optimization techniques. A more sophisticated analysis would involve a nonlinear sensitivity analysis on all potential events or combination of events represented by Δ\vec{S} in a neighborhood of \vec{S} to be defined. Accordingly, the heuristic computer reasoning engine 890 can perform retrodiction, for example:
The telehealth clinic ends much later than expected, can we explain what was the main factors influencing the delay or delays?
Patients' satisfaction is decreasing this month, can we find out why this is different from last month?
There has been no more inconsistency of metric x in patient cognitive test y, what has led to this positive change?

The heuristic computer reasoning engine 890 can perform backcasting (i.e., moving backwards in time, step-by-step, in as many stages as are considered necessary, from the future to the present to reveal the mechanism through which that particular specified future could be attained from the present). The mathematical formulation of that question can be derived from the above one. For example, assuming that

$$∀ j ∈ ( 1 … N ) , \vert {( ℰ ( Δ t ) ( \vec{S} ( t - Δ t ) ) ( \vec{C} ( t - Δ t ) ) )}_{j} \vert < \vec{{ϵ}_{J}} ?$$

one can move one back step further to identify an event that would change the outcome:

Find j∈(1 . . . N),such that |(ε(Δt)(\vec{S}(t−Δt)+ΔS(t−Δt),\vec{C}(t−Δt)))ⱼ|>>\grave{ϵ}ⱼ?

or repeat the process backward in time until such event exists. That would assume that the validity of the prediction holds for that many time steps backward, i.e.

$$\vert ℰ ( n Δ t ) ( \vec{S} ( t - n Δ t ) , \vec{C} ( t - n Δ t ) ) - ℰ ( n Δ t ) ( \vec{{S}_{0}} ( t - n Δ t ) , \vec{C} ( t - n Δ t ) ) \vert \leq κ ( {S}_{0} ) ‖ S ( t ) - {S}_{0} ( t ) ‖$$

where n is the number of back steps Δt involved. Accordingly, the heuristic computer reasoning engine 890 can perform backcasting, for example:
How early could we have predicted that ptosis of patient x is increasing linearly? How early could we have predicted that provider X's performance metrics x and y were impacting telehealth results? How early could we have anticipated to release the staff early, or altogether, from their duties for the day?)

FIG. 9 illustrates the practitioner user interface 900 according to exemplary embodiments.

As shown in FIG. 9, the practitioner user interface 900 may include patient video data 644 showing a view of the patient 101, practitioner video data 614 showing a view of the practitioner 102, and patient system controls 160 (e.g., to control the volume of the patient video data 644, control the patient camera 260 to capture a region of interest 603, etc. In the embodiment of FIG. 9, the practitioner user interface 900 also includes a workflow progression 930, which provides a graphic representation of the workflow progress (e.g., a check list, a chronometer, etc.). Additionally, the practitioner user interface 900 provides a flexible and adaptive display of patient metrics 950 (e.g., sensor data 740 and/or state variables 810).

As described above, construction of a digital twin 800 model of the telehealth patient 101 supports heuristic computer reasoning 890 in the specific medical problem domain as an adjoint to the telehealth workflow and reporting that the practitioner 102 is handling. Meanwhile, similar to a modern cockpit of a fighter jet (that can assist the pilot to focus on his objective, gathering lateral information that may have escaped his attention and supporting flight conditions semi-automatically in order to lower the cognitive load of the pilot), the practitioner user interface 900 provides a “smart and enhanced cockpit” capability that manages information that could otherwise overload the practitioner 102 with information that is not needed.

The server 180, the physician system 120, the microcomputer 310 of the control box 300, and the compact computer 510 of the patient computing system 500 may be any hardware computing device capable of performing the functions described herein. Accordingly, each of those computing devices includes non-transitory computer readable storage media for storing data and instructions and at least one hardware computer processing device for executing those instructions. The computer processing device can be, for instance, a computer, personal computer (PC), server or mainframe computer, or more generally a computing device, processor, application specific integrated circuits (ΔSIC), or controller. The processing device can be provided with, or be in communication with, one or more of a wide variety of components or subsystems including, for example, a co-processor, register, data processing devices and subsystems, wired or wireless communication links, user-actuated (e.g., voice or touch actuated) input devices (such as touch screen, keyboard, mouse) for user control or input, monitors for displaying information to the user, and/or storage device(s) such as memory, RAM, ROM, DVD, CD-ROM, analog or digital memory, database, computer-readable media, and/or hard drive/disks. All or parts of the system, processes, and/or data utilized in the system of the disclosure can be stored on or read from the storage device(s). The storage device(s) can have stored thereon machine executable instructions for performing the processes of the disclosure. The processing device can execute software that can be stored on the storage device. Unless indicated otherwise, the process is preferably implemented automatically by the processor substantially in real time without delay.

The processing device can also be connected to or in communication with the Internet, such as by a wireless card or Ethernet card. The processing device can interact with a website to execute the operation of the disclosure, such as to present output, reports and other information to a user via a user display, solicit user feedback via a user input device, and/or receive input from a user via the user input device. For instance, the patient system 200 can be part of a mobile smartphone running an application (such as a browser or customized application) that is executed by the processing device and communicates with the user and/or third parties via the Internet via a wired or wireless communication path.

The system and method of the disclosure can also be implemented by or on a non-transitory computer readable medium, such as any tangible medium that can store, encode or carry non-transitory instructions for execution by the computer and cause the computer to perform any one or more of the operations of the disclosure described herein, or that is capable of storing, encoding, or carrying data structures utilized by or associated with instructions. For example, the database 182 is stored is non-transitory computer readable storage media that is internal to the server 180 or accessible by the server 180 via a wired connection, a wireless connection, a local area network, etc.

The heuristic computer reasoning engine 890 may be realized as software instructions stored and executed by the server 180.

In some embodiments, the sensor data classification module 720 may be realized as software instructions stored and executed by the server 180, which receives the sensor data 740 captured by the patient computing system 500 and data (e.g., input by the physician 102 via the physician user interface 900) from the physician computing system 102. In preferred embodiments, however, the sensor data classification module 720 may be realized as software instructions stored and executed by the patient system 200 (e.g., by the compact computer 510 of the patient computing system 500). In those embodiments the patient system 200 may classify the sensor data 740 (e.g., as belonging to one of a number of predetermined ranges and/or including any of a number of predetermined patterns) using algorithms (e.g., lower level artificial intelligence algorithms) specified by and received from the server 180.

Analyzing the sensor data 740 at the patient computing system 500 provides a number of benefits. For instance, the sensor data classification module 720 can accurately time stamp the sensor data 740 without being affected by any time lags caused by network connectivity issues. Additionally, analyzing the sensor data 740 at the patient computing system 500 enables the sensor data classification module 720 to analyze the sensor data 740 at its highest available resolution (e.g., without compression) and eliminates the need to transmit that high resolution sensor data 740 via the communications networks 170. Meanwhile, by analyzing the sensor data 740 at the patient computing system 500 and transmitting state variables 810 to the server 180 (e.g., in encrypted form), the cyber-physical system 100 may address patient privacy concerns and ensure compliance with regulations regarding the protection of sensitive patient health information, such as the Health Insurance Portability and Accountability Act of 1996 (HIPAA).

While preferred embodiments have been described above, those skilled in the art who have reviewed the present disclosure will readily appreciate that other embodiments can be realized within the scope of the invention. Accordingly, the present invention should be construed as limited only by any appended claims.
¹ Automatically document care with the Dragon Ambient experience, Ambient Clinical Intelligence system, https://www.nuance.com/healthcare/ambient-clinical-intelligence.html#in-action
² See, e.g., M. Garbey, N. Sun, A. Merla, and I. Pavlidis, Contact-Free Measurement of Cardiac Pulse Based on the Analysis of Thermal Imagery, IEEE Transactions on Biomedical Engineering journal, vol. 54, no. 8, August 2007: I.Pavlidis, J.Dowdall, N.Sun, C. Puri, J.Fei and M.Garbey, Interacting with Human Physiology, Computer Vision and Image Understanding, vol. 108, no. 1-2, October/November 2007.
³ See, e.g., Procházka, Aleš et al. “Breathing Analysis Using Thermal and Depth Imaging Camera Video Records.” Sensors (Basel, Switzerland) vol. 17,6 1408. 16 Jun. 2017, doi: 10.3390/s17061408
⁴ See The status of computerized cognitive testing in aging: A systematic review, Katherine Wild, Ph.D., Diane Howieson, Ph. D., [ . . . ], and Jeffrey Kaye, M. D. Alzheimers Dement. 4(6), pp 428-437, 2008
⁵ See, e.g., Perinasal imaging of physiological stress and its affective potential, Dvijesh Shastri, Manos Papadakis, Panagiotis Tsiamyrtzis, Barbara Bass, Ioannis Pavlidis, IEEE Transactions on Affective Computing, Vol 3 Issue 3, Oct. 22, 2012: M. Garbey, N. Sun, A. Merla, and I. Pavlidis, Contact-Free Measurement of Cardiac Pulse Based on the Analysis of Thermal Imagery, IEEE Transactions on Biomedical Engineering journal, vol. 54, no. 8, August 2007: I.Pavlidis, J.Dowdall, N.Sun, C. Puri, J.Fei and M.Garbey, Interacting with Human Physiology, Computer Vision and Image Understanding, vol. 108, no. 1-2, October/November 2007.
⁶ See, e.g., Review: When I Look into Your Eyes: A Survey on Computer Vision Contributions for Human Gaze Estimation and Tracking, Dario Cazzato, Marco Leo, and Cosimo Distante and Holger Voos, Sensors MDPI, 3 Jul. 2020.
⁷ See, e.g., Emotion recognition using facial expressions, Pawel Tarnowski, Marcin Kołodziej, Andrzeij Majkowski, Remigiusz J.Rak, Procedia Computer Science, Volume 108, 2017, Pages 1175-1184, Procedia Computer Science.
⁸ See, e.g., Using Computer Vision and Machine Learning to Monitor Activity While Working from Home, An introduction to building vision-based health monitoring software on embedded systems, Raymond Lo, Apr. 24, 2020, https://towardsdatascience.com/using-cv-and-ml-to-monitor-activity-while-working-from-home-f59e5302fe67: Survey on Emotional Body Gesture Recognition, Fatemeh Noroozi, Dorota Kaminska, Ciprian Adrian Corneanu, Tomasz Sapinski, Sergio Escalera, and Gholamreza Anbarjafari, https://arxiv.org/pdf/1801.07481.pdf
⁹ See, e.g., Speech Emotion Identification Using Linear Predictive Coding and Recurrent Neural Muhammad Yusup Zakaria, E. C. Djamal, Fikri Nugraha, Fatan Kasyidi, Computer Science 2020, 3rd International Conference on Computer and Informatics Engineering (IC2IE).
¹⁰ See Valentin Bazarevsky, Ivan Grishchenko, Karthik Raveendran, Tyler Zhu, Fan Zhang, Matthias Grundmann, BlazePose: On-device Real-time Body Pose tracking, arXiv: 2006.10204v1 [cs.CV], https://doi.org/10.48550/arXiv.2006.10204.
¹¹ See, e.g., V. Jain et E. Learned-Miller, FDDB: A Benchmark for Face Detection in Unconstrained Settings, p. 11: A. T. Kabakus, An Experimental Performance Comparison of Widely Used Face Detection Tools, ADCAIJ Adv. Distrib. Comput. Artif. Intell. J., vol. 8, no 3, p. 5-12, sept. 2019, doi: 10.14201/ADCAIJ201983512
¹² OpenCV Haar Cascade Eye detector.

www.github.com/opencv/opencv/blob/master/data/haarcascades/haarcascade_eye.xml
¹³ M. H. An, S. C. You, R. W. Park, et S. Lee, Using an Extended Technology Acceptance Model to Understand the Factors Influencing Telehealth Utilization After Flattening the COVID-19 Curve in South Korea: Cross-sectional Survey Study, JMIR Med. Inform., vol. 9, no 1, p. e25435, janv. 2021, doi: 10.2196/25435
¹⁴ See, e.g., Review: When I Look into Your Eyes: A Survey on Computer Vision Contributions for Human Gaze Estimation and Tracking, Dario Cazzato, Marco Leo, and Cosimo Distante and Holger Voos, Sensors MDPI, 3 Jul. 2020.: Stacy V. Smith, Andrew G. Lee, Update on Ocular Myasthenia Gravis, Review Article, Vol. 35, Issue 1, pp. 115-123, February 2017, https://doi.org/10.1016/j.ncl.2016.08.008
¹⁵ See, e.g., G. Liu, Y. Wei, Y. Xie, J. Li, L. Qiao and J.-J. Yang, “A computer-aided system for ocular myasthenia gravis diagnosis,” in Tsinghua Science and Technology, vol. 26, no. 5, pp. 749-758, October 2021, doi: 10.26599/TST.2021.9010025; Stacy V. Smith, Andrew G. Lee, Update on Ocular Myasthenia Gravis, Review Article, Vol. 35, Issue 1, pp. 115-123, February 2017, https://doi.org/10.1016/j.ncl.2016.08.008
¹⁶ See, e.g., Eye Segmentation Method for Telehealth: Application to Myasthenia Gravis Diagnostic, Quentin Lesport, Guillaume Joerger, Henry J. Kaminski, Helen Girma, Sienna McKnett, Marc Garbey, submitted to Artificial Intelligence in Medicine on Aug. 30, 2022
¹⁷ See, e.g., Celakil T, Özcan M. Evaluation of reliability of face scanning using a new depth camera. Int J Esthet Dent. 2021 Aug. 17: 16 (3): 324-337. PMID: 34319667.
¹⁸ See McConnochie K M, Ronis S D, Wood N E, Ng P K. Effectiveness and Safety of Acute Care Telemedicine for Children with Regular and Special Healthcare Needs. Telemed J E Health. 2015: 21 (8): 611-21.
¹⁹ Luboz V, Promayon E, Payan Y. Linear elastic properties of the facial soft tissues using an aspiration device: towards patient specific characterization. Ann Biomed Eng. 2014 November: 42 (11): 2369-78. Doi: 10.1007/s10439-014-1098-1. Epub 2014 Sep. 4. PMID: 25186433
²⁰ E.g., Jain H. P., Subramanian A., Das S., Mittal A. (2011) Real-Time Upper-Body Human Pose Estimation Using a Depth Camera. In: Gagalowicz A., Philips W. (eds) Computer Vision/Computer Graphics
²¹ See, e.g., Procházka, Aleš et al. “Breathing Analysis Using Thermal and Depth Imaging Camera Video Records.” Sensors (Basel, Switzerland) vol. 17,6 1408. 16 Jun. 2017, doi: 10.3390/s17061408
²² See, e.g., Abdel-Ouahab Boudraa, Fabien Salzenstein, Teager-Kaiser energy methods for signal and image analysis: A review. Digital Signal Processing 78, 338-375, 2018
²³ See, e.g., Pan, Y. N., J. Chen, and X. L. Li. “Spectral Entropy: A Complementary Index for Rolling Element Bearing Performance Degradation Assessment.” Proceedings of the Institution of Mechanical Engineers, Part C: Journal of Mechanical Engineering Science. Vol. 223, Issue 5, 2009, pp. 1223-1231: Sharma, V., and A. Parey. “A Review of Gear Fault Diagnosis Using Various Condition Indicators.” Procedia Engineering. Vol. 144, 2016, pp. 253-263
²⁴ See, e.g., Shen, J., J. Hung, and L. Lee. “Robust Entropy-Based Endpoint Detection for Speech Recognition in Noisy Environments.” ICSLP. Vol. 98, November 1998
²⁵ See, e.g., Vakkuri, A., A. Yli-Hankala, P. Talja, S. Mustola, H. Tolvanen-Laakso, T. Sampson, and H. Viertio-Oja. “Time-Frequency Balanced Spectral Entropy as a Measure of Anesthetic Drug Effect in Central Nervous System during Sevoflurane, Propofol, and Thiopental Anesthesia.” Acta Anaesthesiologica Scandinavica. Vol. 48, Number 2, 2004, pp. 145-153
²⁶ Jing Z. Liu, Robert W. Brown, Guang H. Yue, A Dynamical Model of Muscle Activation, Fatigue, and Recovery, Biophysical Journal, Volume 82, Issue 5, 2002, Pages 2344-2359, ISSN 0006-3495, https://doi.org/10.1016/S0006-3495 (02) 75580-X
²⁷ S. Casarin, M.Garbey, S. A. Berceli, Linking gene dynamics to vascular hyperplasia: Toward a predictive model of vein graft adaptation, Plos one, Published: Nov. 30, 2017, https://doi.org/10.1371/journal.pone.018760
²⁸ See, e.g., Emotion recognition using facial expressions, Pawel Tarnowski, Marcin Kołodziej, Andrzeij Majkowski, Remigiusz J.Rak, Procedia Computer Science, Volume 108, 2017, Pages 1175-1184, Procedia Computer Science: Using Computer Vision and Machine Learning to Monitor Activity While Working From Home, An introduction to building vision-based health monitoring software on embedded systems, Raymond Lo, PhD, Apr. 24, 2020, https://towardsdatascience.com/using-cv-and-ml-to-monitor-activity-while-working-from-home-f59e5302fe67: Survey on Emotional Body Gesture Recognition, Fatemeh Noroozi, Dorota Kaminska, Ciprian Adrian Corneanu, Tomasz Sapinski, Sergio Escalera, and Gholamreza Anbarjafari, https://arxiv.org/pdf/1801.07481.pdf: Speech Emotion Identification Using Linear Predictive Coding and Recurrent Neural Muhammad Yusup Zakaria, E. C. Djamal, Fikri Nugraha, Fatan Kasyidi, Computer Science 2020, 3rd International Conference on Computer and Informatics Engineering (IC2IE): Jeremiah R. Barr, Kevin W. Bowyer, Patrick J. Flynn and Soma Biswas, Face Recongnition from Video: A Review, International Journal of Pattern Recognition and Artificial Intelligence, Vol. 26, No. 05, 1266002 (2012) Biometrics, https://doi.org/10.1142/S0218001412660024; Youngjun Cho, Simon J. Julier, Nicolai Marquardt, and Nadia Bianchi-Berthouze, Robust tracking of respiratory rate in highdynamic range scenes using mobile thermal Imaging, Biomed Opt Express. 2017 Oct. 1: 8 (10): 4480-4503: M.Garbey, N. Sun, A. Merla, and I. Pavlidis, Contact-Free Measurement of Cardiac Pulse Based on the Analysis of Thermal Imagery, IEEE Transactions on Biomedical Engineering journal, vol. 54, no. 8, August 2007: I.Pavlidis, J.Dowdall, N.Sun, C. Puri, J.Fei and M.Garbey, Interacting with Human Physiology, Computer Vision and Image Understanding, vol. 108, no. 1-2, October/November 2007.
²⁹ See, e.g., Associations between acute exposures to PM2.5 and carbon dioxide indoors and cognitive function in office workers: a multicountry longitudinal prospective observational study, Jose Guillermo Cedeño Laurent et al 2021 Environ. Res. Lett. 16 094047: Low Indoor Temperatures and Morbidity in the Ederly, K. J. Collins, Age and Ageing, Volume 15, Issue 4, July 1986, Pages 212-220, https://doi.org/10.1093/ageing/15.4.212; S. Tham, R. Thompson, O. Landeg, K. A. Murray, T. Waite, Indoor temperature and health: a global systematic review; Public Health, Volume 179, pp 9-17, 2020; Peder Wolkoff, Indoor air humidity, air quality, and health—An overview, International Journal of Hygiene and Environmental Health, Volume 221, Issue 3, pp 376-390, 2018
³⁰ See, e.g., Thermography in Pain Management: A technique for assessing and tracking changes in vascular-related pain syndromes. Richard A. Sherman, PhD, Gabriel Tan, PhD and Bilal F. Shanti, MD, Practical Pain Management, Vol 4 Issue 4, 2012.
³¹ See, e.g., Wireless Presence Check System, U.S. Prov. Pat. Appl. No. 63/104,865, Marc Garbey and Shannon Furr.
³² Vadivelu S., Ganesan S., Murthy O. V. R., Dhall A. (2017) Thermal Imaging Based Elderly Fall Detection. In: Chen C S., Lu J., Ma K K. (eds) Computer Vision-ΔCCV 2016 Workshops. ΔCCV 2016. Lecture Notes in Computer Science, vol 10118. Springer, Cham. https://doi.org/10.1007/978-3-319-54526-4 40
³³ Björnsson, B., Borrebaeck, C., Elander, N. et al. Digital twins to personalize medicine. Genome Med 12, 4 (2020). https://doi.org/10.1186/s13073-019-0701-3
³⁴ Elisa Negri (2017). “A review of the roles of Digital Twin in CPS-based production systems”. Procedia Manufacturing. 11:939-948
³⁵ E. Bonabeau, Agent-based modeling: Methods and techniques for simulating human systems, Proceedings of the National Academy of Sciences of the United States of America. 99:7280-7. May 14, 2002: Grimm, Volker: Railsback, Steven F., Individual-based Modeling and Ecology, Princeton University Press. p. 485. ISBN 978-O-691-09666-7. 2005: Bruynseels, Koen: Santoni de Sio, Filippo: van den Hoven, Jeroen (February 2018). “Digital Twins in Health Care: Ethical Implications of an Emerging Engineering Paradigm”. Frontiers in Genetics. 9:31. doi: 10.3389/fgene.2018.00031. PMC 5816748. PMID 29487613.
³⁶ M Garbey, S Casarin, S A Berceli, Vascular Adaptation: Pattern Formation and Cross Validation between an Agent Based Model and a Dynamical System, Journal of Theoretical Biology 429, 149-163, 2017; S. Casarin, M.Garbey, S. A. Berceli, Linking gene dynamics to vascular hyperplasia, Toward a predictive model of vein graft adaptation, Plos one, Published: Nov. 30, 2017, https://doi.org/10.1371/journal. pone. 018760: M. Garbey, M. Rahman and S. Berceli, A Multiscale Computational Framework to Understand Vascular Adaptation, Journal of Computational Science Volume 8, May 2015, Pages 3247.
³⁷ See, e.g., Leanne M. Hirshfield et Al, Using Noninvasive Brain Measurement to Explore the Psychological Effects of Computer Malfunctions on Users during Human-Computer Interactions, Advances in Human-Computer Interaction, 2014, Article ID 101038 https://doi.org/10.1155/2014/101038; Haleh Aghajani, Marc Garbey & Ahmet Omurtag, Measuring Mental Workload with EEG+fNIRS, Frontiers in Human Neuroscience 11 (2017)
³⁸ See the extensive list of publications by Rosalind W Picard
³⁹ See, e.g., Bae J M. The clinical decision analysis using decision tree. Epidemiol Health. 2014 Oct. 30:36:e2014025. doi: 10.4178/epih/e2014025. PMID: 25358466; PMCID: PMC4251295: Aleem I S, Schemitsch E H, Hanson B P. What is a clinical decision analysis study? Indian J Orthop. 2008 April: 42 (2): 137-9. doi: 10.4103/0019-5413.40248. PMID: 19826517: PMCID: PMC2759613.
⁴⁰ See, e.g., Chen T, Wang Y, Chen H, Marder K, Zeng D. Targeted local support vector machine for age-dependent classification. J Am Stat Assoc. 2014:109:1174-1187.
⁴¹ See, e.g., Breiman L. Random forests. Mach Learn. 2001:45:5-32
⁴² See, e.g., Hinton G E, Osindero S, Teh Y W. A fast learning algorithm for deep belief nets. Neural Comput. 2006:18:1527-1554.

1. A cyber-physical system for conducting a telehealth session between a practitioner and a patient, the system comprising:
a practitioner system, comprising:
a practitioner camera configured to capture practitioner video data of the practitioner;
a practitioner microphone configured to capture practitioner audio data of the practitioner; and
a practitioner display configured to display patient video data via a practitioner user interface; and
a practitioner speaker configured to output patient audio data;
a patient system, in network communication with the practitioner system, comprising:
a patient display configured to display the practitioner video data;
a patient speaker configured to output the practitioner audio data;
a patient microphone configured to capture the patient audio data;
a patient camera configured to capture the patient video data; and
a hardware control box that includes hardware buttons and provides functionality for the patient to initiate the telehealth session.

2. The system of claim 1, wherein the system provides functionality for the patient to initiate the telehealth session with the practitioner via:
a single click of one of the hardware buttons; or
a voice command, input via the patient microphone, that is recognized by the system using voice recognition.

3. (canceled)

4. The system of claim 1, wherein:
the control box includes a beeper configured to output an audible sound;
the system provides functionality for the practitioner to activate the beeper to help the patient locate the control box.

5. The system of claim 4, wherein the beeper is configured to output the audible sound via the patient speaker, the system further comprising:
an audio calibration module configured to:
receive audio data, captured by the patient microphone, indicative of the audible signal output by the beeper via the patient speaker;
adjust the volume of the patient speaker or the sensitivity of the patient microphone based on the received audio data.

6. The system of claim 1, wherein the patient camera is enclosed in a camera enclosure, the camera enclosure comprising a mirror that enables the patient camera to capture patient video data of the patient and prevent the patient from seeing the patient camera.

7. The system of claim 1, wherein the patient system further comprises one or more environmental sensors that capture information indicative of one or more environmental conditions.

8. The system of claim 1, further comprising:
a sensor data classification module configured to analyze sensor data captured by the patient system and calculate one or more state variables indicative of the physical, emotive, cognitive, or social state of the patient.

9. The system of claim 8, wherein:
the patient system further comprises an eye tracker, a thermal imaging camera, or a depth camera; and
the sensor data classification module is configured calculate the one or more state variables based on eye tracking data captured by the eye tracker, thermal images captured by the thermal imaging camera, or three-dimensional images captured by the depth camera.

10. The system of claim 8, wherein:
the patient system is configured to conduct a computer-assisted cognitive impairment assessment by:
outputting questions for the patient via the patient display;
providing functionality for the patient to provide responses to the questions using the hardware buttons of the control box; and
time stamping the questions output via the patient display and the responses provided by the patient; and
the sensor data classification module is configured to calculate state variables indicative of the cognitive state of the patient based on the time-stamped questions output via the patient display and the time-stamped responses provided by the patient.

11. The system of claim 8, wherein the sensor data classification module is configured to perform audio analysis on the patient audio data to calculate one or more state variables indicative of the physical, emotive, cognitive, or social state of the patient.

12. The system of claim 8, wherein the sensor data classification module is configured to perform computer vision analysis on the patient video data to calculate one or more state variables indicative of the physical, emotive, cognitive, or social state of the patient.

13. The system of claim 12, wherein the sensor data classification module calculates one or more state variables indicative of a neurological disease.

14. The system of claim 8, wherein the practitioner user interface displays:
the patient video data captured by the patient camera; and
the sensor data captured by the patient system or the one or more state variables calculated by the sensor data classification module.

15. The system of claim 8, wherein the one or more state variables are combined with previously-determined state variables to form a digital twin, the digital twin comprising a mathematical representation of the physical, emotive, cognitive, or social state of the patient.

16. (canceled)

17. The system of claim 16, wherein the patient camera is a remotely-controllable pan-tilt-zoom camera, and further comprising:
a computer vision module configured to perform computer vision analysis to identify each region of interest in the patient video data; and
a patient tracking module configured to output control signals to the pan-tilt-zoom camera to zoom in on a region of interest relevant to the examination being performed.

18. The system of claim 17, wherein the computer vision module uses the digital twin of the patient to identify the regions of interest in the patient video data.

19. The system of claim 18, further comprising a heuristic computer reasoning engine configured to:
detect deviations between the one or more state variables calculated by the sensor data classification module and previously-determined state variables included in the digital twin of the patient; or
identify potentially relevant diagnostic explorations based on the digital twin of the patient and the one or more state variables calculated by the sensor data classification module.

20. A method of conducting a telehealth session between a practitioner and a patient, the method comprising:
capturing patient video data of the patient by a patient camera of a patient system, the patient system comprising a hardware control box that includes hardware buttons;
displaying the patient video data, via a practitioner user interface, by a practitioner display;
capturing practitioner video data of the practitioner by a practitioner camera;
displaying the practitioner video data by a patient display;
capturing practitioner audio data by a practitioner microphone;
outputting the practitioner audio data by a patient speaker;
capturing patient audio data by a patient microphone;
outputting the patient audio data by a practitioner speaker; and
providing functionality for the patient to initiate the telehealth session using the hardware control box.

21. A patient system for a patient to conduct a telehealth session with a practitioner having a practitioner system, the practitioner system including a practitioner microphone that captures practitioner audio data of the practitioner, a practitioner display that displays patient video data via a practitioner user interface, and a practitioner speaker that outputs patient audio data, the patient system comprising:
a patient computing system;
a patient speaker configured to output the practitioner audio data;
a patient microphone configured to capture the patient audio data;
a patient camera configured to capture the patient video data; and
a hardware control box comprising:
one or more patient-actuatable input devices;
a processing unit that that communicates with the patient computing system and outputs an instruction to initiate the telehealth session in response to patient actuation of one of the one or more patient-actuatable input devices.

22. The method of calculating state variables indicative of the physical, emotive, cognitive, or social state of a patient, the method comprising:
receiving patient audio data captured by a patient microphone of a patient system for conducting a telehealth session;
receiving patient video data captured by a patient camera of a patient system for conducting the telehealth session;
performing audio analysis on the patient audio data; and
performing computer vision analysis on the patient video data.

23. The method of claim 22, wherein the state variables are indicative of a neurological disease.

24. The method of claim 22, further comprising:
calculating additional state variables by analyzing eye tracking data captured by an eye tracker, thermal images captured by a thermal imaging camera, or three-dimensional images captured by a depth camera.

25. The method of claim 22, further comprising:
outputting questions for the patient;
time stamping the questions;
receiving patient responses to the questions;
time stamping the patient responses; and
calculating additional state variables indicative of the cognitive state of the patient based on the time-stamped questions and the time-stamped patient responses.
